(19) United States
(12) Patent Application Publication
(10) Pub. No.: US 2012/0296253 A1
(43) Pub. Date: Nov. 22, 2012

(54) HAEMODIALYSIS MACHINE RETROFIT AND CONTROL INSTALLATION AND USE THEREOF FOR THE TREATMENT OF PROLIFERATIVE DISORDERS

(76) Inventors: Edward Henry Mathews, Silver Lakes (ZA); Leon Liebenberg, Silver Lakes (ZA); Ruaan Pelzer, Pretorius Park (ZA)

(21) Appl. No.: 13/513,936

(22) PCT Filed: Dec. 9, 2010
(86) PCT No.: PCT/IB2010/055686

§ 371 Date: Aug. 8, 2012

(30) Foreign Application Priority Data
Dec. 10, 2009 (ZA) 2009/08784

(51) Int. Cl. A61M 1/36

(52) U.S. Cl. 604/4.01

(57) ABSTRACT

A haemodialysis machine retrofit and control installation including an intake-flow blood glucose sensor and an intake-flow blood glutamine sensor, a return-flow blood glucose sensor and return-flow blood glutamine sensor, a dialysate glucose- and glutamine controller, a central control unit connected to the blood glucose- and glutamine sensors, the dialysate glucose- and glutamine controllers for regulating the glucose and glutamine levels in the dialysate to obtain required blood-glucose and glutamine concentrations at the return-flow blood glucose- and glutamine sensors and an electroencephalograph (EEG) monitor providing the central control unit with information pertaining to spontaneous electro-cerebral activity to initiate raising of glucose and glutamine levels. Also disclosed is a method of treating a proliferative disorder in a human or animal, by reducing via a retrofitted haemodialysis machine blood-glucose or glutamine concentrations in the human or animal body for a pre-defined period of time.

### PRIORITY CLAIM

This patent application is a U.S. National Phase of International Patent Application No. PCT/IB2010/055686, filed 9 Dec. 2010, which claims priority to South African Patent Application No. 2009/08784, filed 10 Dec. 2009, the disclosures of which are incorporated herein by reference in their entirety.

### FIELD

The presently disclosed embodiments relate to treatment of proliferative disorders. In particular, the disclosed embodiments relate to a haemodialysis machine retrofit and control installation, a cerebral glycaemic control module, a method for the extracorporeal treatment of blood and use of a haemodialysis machine retrofit and control installation for treating a proliferative disorder in a human or animal.

### BACKGROUND

The publications and other material used herein to illuminate the background of the disclosed embodiments, and, in particular, cases to provide additional details respecting to practice, are incorporated by reference.

### Therapy of Proliferative Disorders

Proliferative disorders are depicted by the uncontrolled growth of cells of certain tissues, and can be classified as cancerous and non-cancerous.

Cancerous proliferative disorders are depicted by the “uncontrolled growth” (division beyond normal limits) of cells with a “malignant” (invasive, destructive) phenotype. Such cells not only form cancerous lesions (tumours), but can invade underlying tissue or migrate to other areas of the body via lymph or blood, i.e., “metastasis” (Hanahan et al. 2000, Gatenby et al. 2004, Kohn et al. 1995, Souhami et al. 2005).

Examples of cancerous proliferative disorders include the various types of carcinoma, sarcoma, lymphoma, leukemia, germ cell tumours, and blastoma. Therapy of cancerous proliferative disorders includes surgical resection, chemotherapy, and radiotherapy (Souhami et al. 2005), as well as lifestyle interventions.

Non-cancerous proliferative disorders are depicted by the uncontrolled growth of cells with a benign phenotype. This implies that the cells evade only normal controls on growth, but cannot metastasize (Gatenby et al. 2004, Kohn et al. 1995).

Examples of non-cancerous proliferative disorders include the following: vascular restenosis in patients with coronary artery disease (Albiero et al. 2000, Adamien et al. 2000), vascular smooth muscle cell (VSMC) proliferation which plays an important role in the development of atherosclerosis and restenosis (Libby et al. 1992), benign proliferative breast disease (Bodian et al. 1993), chronic inflammation (Kundu et al. 2008, Balkwell et al. 2001), progressive multiple sclerosis (Osame 1987), myelodegenerative disease (Osame 1987), neurofibromatosis (Riccardi et al. 1987), keloid formation (Levy et al. 1976), Paget's disease of the bone (Hamdy 1994), fibrosis and cirrhosis (Teli et al. 1995), and eosinophilic granuloma of the bone (Lichtenstein 1964).

Therapy of non-cancerous proliferative disorders includes surgical resection, hormone (e.g., cortisone) therapy, radiation therapy (including laser therapy and brachytherapy), as well as lifestyle interventions (e.g., diet, physical activity, and weight control).

### Increased Glucose Uptake in Non-Cancerous Proliferative Disorders

A revival in tumour bio-energetics was initiated in the mid-1990's when it was discovered that positron emission tomography (PET) imaging using the non-metabolizable glucose analogue 2-[¹⁸F]-2-deoxy-D-glucose (FDG), could detect and map many types of tumours (Shankar et al. 2006, Huang 2000, Zasadny et al. 1993, Kosaka et al. 2008).

The standardized uptake value (SUV) is the semi-quantitative method most commonly used to determine FDG (glucose analogue) uptake in attenuation-corrected PET images (Huang 2000). An SUV=1 indicates uniform distribution of radioactive FDG tracer, whereas SUV>1 indicates FDG accumulation (due to FDG's inability to be metabolized beyond its phosphorylated stage during glycolysis) (Shankar et al. 2006, Huang 2000, Zasadny et al. 1993, Kosaka et al. 2008). FDG accumulates in the tissue at a rate proportional to the rate of glucose utilization (Huang 2000).

Table 1 shows the SUV values for a number of non-cancerous proliferative disorders. It is apparent that non-cancerous proliferative disorders display a marked increase in glucose uptake, with SUV values ranging between 1.4 and 8.4.

TABLE 1
FDG uptake classification in non-cancerous
proliferative disorders (PET-derived data).

| Tissue/Organ | SUV ± σ | Ref. |
|---|---|---|
| Benign uterine leiomyoma | 8.4 | Vriens et al. 2010 |
| Chronic bacterial osteomyelitis | 1.78 | Sahlmann et al. 2004, |
| | | Guhlmann et al. 1998 |
| Neurofibroma (cervical) | 5.3 | Son et al. 2007 |
| Neurofibroma (paratracheal) | 1.8 | Son et al. 2007 |
| Benign bone tumours | 2.18 ± 1.52 | Aoki et al. 2001 |
| Benign breast tumour | 1.4 | Chen 2008, |
| | | Avril et al. 1996 |
| Granuloma | 1.53 ± 0.04 | Zhao et al. 2009 |
| Benign liver granulomatous | 5.0 ± 4.0 | Zu et al. 2004 |
| Experimental inflammation (rat) | 2.33 | Zhuang et al. 2001 |
| Inflammatory bowel disease | >3.0 | Löffler et al. 2006 |
| Carotid plaque inflammation | 2.7 | Arauz et. al. 2007 |
| Non-inflamed plaque in animal | 2.71 | Davies et al. 2010, |
| atherosclerosis | | Bural et al. 2008 |
| Benign lung nodule | 2.37 | Zhuang et al. 2001 |
| Radiation reaction | 2.56 | Zhuang et al. 2001 |
| Painful lower leg prosthesis | 2.61 | Zhuang et al. 2001 |
| Fibrocystic/chronic inflammation | 1.5 | Dehdashti et al. 1995 |

SUV = mean Standardised Uptake Value; σ = standard deviation

The rest of the disclosed embodiments will focus primarily on the treatment of cancerous proliferative disorders, by way of explaining the application of the inventive extracorporeal treatment of blood for patients with either cancerous or non-cancerous disorders.

### Cancerous Proliferative Disorders

Compared to normal tissues, cancer cells have a remarkably different metabolism from that of the tissues from which they are derived (De Berardinis et al. 2009). They exhibit an altered metabolism that allows them to sustain higher proliferative rates and to resist cell-death signals (Jones et al. 2009). This implies that cancer cells are more nutrient hungry and excrete more waste products than their normal tissue counterparts (Kim et al. 2006, Warburg 1956, Vander Heiden et al. 2009, Menendez et al. 2007, Chi et al. 1999, Gatenby et al. 2004), resulting in a build-up of metabolic energy sources inside the cell and the formation of a more hostile environment outside the cell.

In order to divide, cells need to both increase their size and to replicate their DNA, which are hugely metabolically demanding (Seyfried et al. 2010). These processes require large amounts of proteins, lipids and nucleotides, as well as energy in the form of adenosine-triphosphate (ATP). This anabolic drive requires cells to increase their uptake of the building blocks for this process, major nutrients being glucose and amino acids (Jones et al. 2009, Tennant et al. 2010, Seyfried et al. 2010, Feron 2009).

### Glucose as Primary Metabolic Energy Source in Cancerous Proliferative Disorders

For most of their energy needs, normal cells rely on oxidative phosphorylation via the tricarboxylic acid (TCA) cycle (i.e., “respiration”), which consumes oxygen and glucose to produce energy-storing molecules of adenosine-triphosphate (ATP). However, cancer cells typically depend more on glycolysis (i.e., “fermentation”), the anaerobic breakdown of glucose into ATP (Seyfried et al. 2010, Murray et al. 2006, Guppy et al. 1993). Such increased glycolysis, even in the presence of oxygen, is termed the Warburg effect (Kim et al. 2006, Warburg 1956, Vander Heiden et al. 2009).

As with non-cancerous tumours, cancerous tumours can also be detected by FDG-PET imaging. Even more importantly, more than 90% of difficult-to-treat metastatic tumours are hypoxic (Shaw 2006) hence also highly glycolytic (Feron 2009), which results in their accurate detection using FDG-PET scanning (Shankar et al. 2006, Huang 2000, Zasadny et al. 1993, Kosaka et al. 2008).

FDG-PET imaging also suggests that the glycolytic switch precedes the angiogenic switch (Jones and Thompson 2009). Researchers have further shown that the high-glycolytic metabolism characteristic expands as cancer cells become more malignant (Tennant et al. 2010, Shankar et al. 2006). These findings further accentuate the crucial importance of glucose as a metabolic energy source for cancer cells.

During cancer progression, the tumour outgrows the local blood supply (Jain 2009, Mankoff et al. 2009, Vaupel 2004), cf. FIG. 1. This ineffective micro-vasculature leads to a drop in local oxygen concentration (viz. hypoxia) and inadequate supply of nutrients (Jones et al. 2009, Mankoff et al. 2009, Vaupel 2004).

The clinically aggressive, more advanced cancer types react to this energy crisis and hostile environment by (among others) activating certain transcription factors, including the hypoxia inducible factor 1 (HIF-1). This protein raises levels of glycolytic enzymes, among others, which helps facilitate an increase in glucose metabolism (via glycolysis) despite poor vascularity, resulting in cancer cell progression, local growth, or metastases (Vaupel 2004), cf. FIG. 1. It also suppresses the body's immune system in the hypoxic region which further promotes angiogenesis and cancer cell invasiveness (Jones et al. 2009, Vaupel 2004).

Other pro-survival signaling proteins, such as Akt, can also convert cancer cells to start using glycolysis. Akt induces glucose transporters to absorb glucose into the cell, and also raises the levels of glycolytic enzymes, among others, which further boost cancer cell metabolism. The shift from respiration to glycolysis apparently affords cancer cells a higher metastatic potential (Jones and Thompson 2009). Increased glycolysis is now accepted for its importance in sustaining tumours, rather than inducing them (Garber 2006).

Table 2 shows the SUV values of some cancerous proliferative disorders. Column 4 of Table 2 shows that most cancers have much larger uptake values of glucose analogue (FDG) than their normal-cell counterparts. The tumour/normal cell ratios vary between 1.7 and 16.8. This provides in vivo proof of the increased glucose metabolism in neoplastic tissue versus that of surrounding normal cells.

TABLE 2
Semi-quantitative assessment of FDG (glucose analogue) uptake in cancer
tissues (PET-derived data); 60-minute to 90-minute uptake times.

| | | | Tumour | |
|---|---|---|---|---|
| | | | SUV/normal | |
| | Pathalogic diagnosis/ | | cell SUV | |
| Host tissue | tumour cell type | SUV ± σ | ratio^{a} ± σ | Reference |
| Liver | Liver metastases | 7.8 ± 4.5 | 4.0 ± 2.0 | Zu et al. 2004, |
| | | | | Delbeke et al. 2009, |
| | | | | Vaupel et al. 1989 |
| | Hepatocellular carcinoma | 5.5 ± 3.9 | 3.0 ± 2.0 | Zu et al. 2004, |
| | | | | Delbeke et al. 2009, |
| | | | | Vaupel et al. 1989, |
| | | | | Chin et al. 2008 |
| Brain | Lymphoma | 22.2 ± 5.0 | 5.6 ± 1.7^{b} | Chin et al. 2008 |
| | Glioma, meningioma, | 11.6 ± 3.7 | 3.1 ± 0.8^{b} | Dastidar et al. 1989 |
| | medulloblastoma, | | | |
| | oligodendroglioma | | | |
| | Metastasis | 7.8 ± 2.7 | 2.6 ± 1.14^{b} | Geschwind et al. |
| | | | | 2002 |
| Lung | Non-small cell lung cancer | 10.1 | 15.8 | Wong et al. 2007 |
| | Metastasis | 10.7 | 16.8 | Weber et al. 2003, |
| | | | | Lin et al. 2003 |
| Mammary gland | Breast cancer | 7.9 ± 5.6 | 2.0 to 2.8 | Tateishi et al. 2008, |
| | | | | Kallinowski et al. |
| | | | | 1989, Kenny et al. |
| | | | | 2005 |
| Colon | Colorectal metastatis | 7.4 | 1.7 to 8.1 | Zu et al. 2004, |
| | | | | Mankoff et al. 2001, |
| | | | | Bystrom et al. 2009, |
| | | | | Cascini et al. 2007, |
| | | | | Schiepers et al. 1998, |
| | | | | Fanciulli et al. 1993 |
| Prostate | Prostate cancer | 7.0 | 1.7 to 2.6 | Jadvar et al. 2008, |
| | | | | Wang et al. 2007 |
| Pancreas | Pancreatic cancer | 8.0 ± 4.9 | 2.4 to 3.3 | Komar et al. 2009 |

SUV = mean of the Standardised Uptake Values*; σ = standard deviation)
*The standardised uptake value (SUV) is the semi-quantitative method most commonly used to determine FDG (glucose analogue) uptake in attenuation-corrected PET images (Huang 2000). An SUV = 1 indicates uniform distribution of radioactive FDG tracer, whereas SUV > 1 indicates FDG accumulation (due to FDG's inability to be metabolised beyond its phosphorylated stage during glycolysis) (Shankar et al. 2006, Huang 2000, Zasadny et al. 1993, Kosaka et al. 2008). FDG accumulates in the tissue at a rate proportional to the rate of glucose utilization (Huang 2000).
^{a}Tumour SUV relative to normal cell SUV, (Wang et al. 2007).
^{b}Ratio of count of maximum pixels in tumour, to average count per pixel in white matter (Kosaka et al. 2008)

### Glutamine as Secondary Metabolic Energy Source in Cancerous Proliferative Disorders

Compared to normal cells, cancer cells also show increased use of glutamine (Seyfried et al. 2005, Seyfried et al. 2010, Balinsky et al. 1984, Briscoe et al. 1994, Molina et al. 1995, Tennant et al. 2010). In cancer cells with functional respiration (viz, normoxic cell volume), glutamine supplies at least 10% of metabolic energy (Gray et al. 1953, Gatenby et al. 2004).

Also, for tumours with defective respiration (highly glycolytic, hypoxic tumours), glutamine oxidation produces significant energy through substrate level phosphorylation in the TCA cycle (Seyfried et al. 2010). This occurs through the action of succinyl-CoA synthetase (SCS) (Seyfried et al. 2010). SCS is the only mitochondrial enzyme capable of ATP production via substrate level phosphorylation in the absence of respiration (Seyfried et al. 2010).

Apart from glutamine fuelling cancer cells, glutaminolysis further provides an important anapleurotic mechanism for replenishing the TCA cycle intermediates that are necessary precursors for the anabolic processes required for cancer cell growth (Feron 2009, Seyfried et al. 2010, Fidler 2003, Tennant et al. 2010, Kaadige et al. 2009, DeBerardinis et al. 2010). Additionally, the NADPH produced by glutaminolysis may act as an energy source to support fatty acid and nucleotide synthesis (Tennant et al. 2010, DeBerardinis et al. 2010).

It follows that cells that convert glucose and glutamine into biomass most efficiently will proliferate fastest (Vander Heiden et al. 2009). Therapies that decrease plasma glucose and glutamine concentrations may well rapidly induce tumour regression, owing to their importance as energetic substrates (Tenant et al. 2010, Jones et al. 2009, Simons et al. 2009, Seyfried et al. 2010).

Ineffectiveness of Chemoradiotherapies with Hypoxic Tumours

FIG. 1 shows that the normoxic cell volume of tumours (situated close to blood supply, resulting in higher levels of oxygen) reacts better to cytotoxic- and radiotherapy than the hypoxic cell volume further away from the blood supply (Vaupel 2004, Gray et al. 1953, Moreno-Sanchez et al. 2007, Seyfried et al. 2005). The reason is that most cytotoxic pharmaceutical compositions and radiotherapies require the presence of oxygen (Mankoff et al. 2009, Vaupel 2004, Gray et al. 1953, Moreno-Sanchez et al. 2007, Seyfried et al. 2005, Seyfried et al. 2010).

FIG. 1 further shows a highly suppressed immune response for the hypoxic region (Jones and Thompson 2009, Mankoff et al. 2009, Kroemer et al. 2008, Moreno-Sanchez et al. 2007). The volume of this region in advanced staging of tumours is also typically several-fold larger than that of its normoxic counterpart. This further complicates effective treatment using only chemoradiotherapies.

As chemoradiotherapy is less effective in hypoxic, solid tumours, they are usually surgically resected. This can restore vascularity (and oxygen supply) (Jain 2009, Wheatley et al. 2005), to make for more effective cytotoxic- and radiotherapy. These surgical procedures are unfortunately often accompanied by cancer cell leakage (Zirngibl et al. 2005) and consequential metastases (Fidler 2008). Often it becomes impractical to surgically remove these hypoxic metastases, which accentuates the need for alternative adjuvant approaches.

The difficult-to-treat solid tumours and metastases with their hypoxic phenotype, therefore present a potentially important therapeutic treatment opportunity via their “rapacious uptake of glucose” (Vousden et al. 2009) and their excessive usage of glutamine (Seyfried et al. 2010). Glucose and glutamine deprivation treatment may therefore complement traditional therapies, due to their proven apoptotic abilities (Seyfried et al. 2005, Seyfried et al. 2010, Lee et al. 1997).

### Glucose and Glutamine Restriction

Chronic restriction of glucose and glutamine energy sources is associated with a switch from anabolic processes (e.g. cell division) to catabolic processes (e.g. cell maintenance and repair) (Howell et al. 2009). Inhibition of the induced anabolic changes in tumours or stimulation of reduced catabolic changes can result in cessation of tumour growth (Seyfried et al. 2005, Tennant et al. 2010, Kaadige et al. 2009, Howell et al. 2009, Martin et al. 2001, Simons et al. 2009, Yuneva et al. 2007).

Tumour cells are less adaptable than normal cells to abrupt changes in metabolic environment and can be either destroyed outright or isolated metabolically from normal cells (Seyfried et al. 2005, Seyfried et al. 2010, Zirngibl et al. 2005). The genomic and metabolic flexibility of normal cells can thus be used to target indirectly the genetically defective and less metabolically flexible tumour cells (Seyfried et al. 2005, Seyfried et al. 2010, Zirngibl et al. 2005). Therefore, this strategy may potentially be employed in treating patients with high-glycolytic tumours (Seyfried et al. 2010, Zirngibl et al. 2005, Wheatley et al. 2005).

When cancer cells are engaged in high-flux aerobic glycolysis, they become addicted to glucose (Garber 2006). “If the glucose is suddenly taken away, their ability to do high-flux glucose capture and metabolism disappears, and the cancer cell has no choice but to die.” (Jones and Thompson 2009). It has been found that glucose withdrawal (and subsequent ATP depletion) induces cell death in a manner indistinguishable from that seen upon withdrawal of growth factor signalling, particularly in a hypoxic environment (Jones and Thompson 2009, Seyfried et al. 2010, Zu et al. 2004, Vander Heiden et al. 2009, Guppy et al. 1993, Jelluma et al. 2006, Lin et al. 2003). Where this has been examined in cancer patients, response to therapy is predicted by the ability to disrupt glucose metabolism as measured by FDG-PET scanning (Vander Heiden et al. 2009).

When deprived of glucose, but with adequate glutamine available, cancer cell survival decreases exponentially in vitro under normoxic conditions, with significant cytotoxicity typically after 2 to 4 hours (Seyfried et al. 2010). After 8 hours upon 100% deprivation of glucose but with adequate glutamine, only 25% of cancer cells survive (Dang et al. 1999, Wise et al. 2008, Yuneva et al. 2007), cf. Table 3.

Other research (normoxic, in vitro) shows that glucose utilisation stops when glutamine availability is restricted (cf. Table 3). This essentially halts cell growth and results in cell death (Dang et al. 1999, Wise et al. 2008, Yuneva et al. 2007).

TABLE 3
Survival in 4-, 12-, and 24-hour glucose-deprived cancer cells (in
vitro), supplemented with glutamine under normoxic conditions

| | Survival^{a} (%) | Reference |
|---|---|---|
| With glucose, after 4, | 100 | Seyfried et al. 2005, |
| 12, and 24 hours | | Kaadige et al. 2009 |
| Without glucose, with | 25 | Seyfried et al. 2005, |
| glutamine after 4 hours | | Yuneva et al. 2007 |
| With glucose, with | 120 | Kaadige et al. 2009 |
| glutamine, after 24 hours | | |
| With glucose, without | 20 | Kaadige et al. 2009, |
| glutamine, after 12 hours | | Yuneva et al. 2007 |
| Without glucose, without | 0 | Kaadige et al. 2009 |
| glutamine, after 12 hours | | |

^{a}Percent survival is normalised to the respective control (“with glucose”)

If one can restrict the body from glucose whilst providing for the absolute minimum cerebral demand, one may go below the minimum amount of BG required to sustain certain cancers. The human brain accounts for 20-25% of resting metabolism, requiring typically 100 to 150 g of glucose per day, (subject-dependent), (Casazza et al. 1984, Siegel et al. 1998).

To provide for this fairly constant 24-hour cerebral glucose consumption, under normal conditions the body ingests food at certain times, stores the resultant BG, and releases it in a controlled manner over 24 hours. The available BG for cancer cells over 24 hours is therefore on average larger than that needed by the brain (Cryer 2007, Auer 2004, Choi et al. 2001, Gruetter et al. 1998). This mean BG concentration for the average human over 24 hours (also available to the cancer cells) is 6 mmol/l (or 110 mg/dl), (Champe et al. 2008, Murray et al. 2006).

When instantaneous body BG concentration is reduced below 2.1 mmol/l, (equivalent to brain glucose concentration of 0.2 μmol/g) (Öz et al. 2009, Öz et al. 2007), the cerebral blood flow increases sharply, indicating the triggering of a defense mechanism aimed at improving glucose delivery to the brain during hypoglycaemia (Öz et al. 2009, Öz et al. 2007). As BG levels drop to the range of 1 to 2 mmol/l in the conscious patient, clinical stupor or drowsiness sets in (Cahill et al. 1966).

When instantaneous BG falls to 1.2 mmol/l, the brain glucose concentration approximates 0 μmol/g (Öz et al. 2009). Such severe hypoglycaemia causes brain energy source deprivation and, as a result, functional brain failure (Öz et al. 2009, Öz et al. 2007). In practice, if BG levels fall below 1 mmol/l for an extended period of time (i.e. >20 min.), depending on body glycogen reserves, neuronal death is assumed to occur (Suh et al. 2007, Auer 2004, Agardh et al. 1980, Cahill et al. 1966). Hypoglycaemic brain injury can be prevented by ensuring continuous spontaneous electro-cerebral activity as monitored by electro-encephalography (EEG), (Auer 2004, Agardh et al. 1980).

The human body can produce about 15% to 30% of the glucose to supply brain needs from protein and glycerol conversion (Lin et al. 2003). However, most brain energy during prolonged fasting is derived from the metabolism of ketone bodies (especially from β-hydroxybutyrate) which are produced from stored fat in the liver (Seyfried et al. 2005, Seyfried et al. 2010, Geschwind et al. 2002, Cahill et al. 1966, Patel et al. 2004, Mantis et al. 2004, Pan et al. 2000). Other organs will metabolise fatty acids as well as ketones for energy, whilst reserving most of the circulating glucose for the brain (Seyfried et al. 2005, Seyfried et al. 2010, Tennant et al. 2010).

Furthermore, Seyfried and co-workers have shown that the elevation of blood ketone levels through the adoption of a low-calorie (e.g. 400 to 500 kcal per day) high-ketogenic diet (e.g. 4:1, fat:carbohydrate+protein) for three to five days can protect the brain from oxidative stress at low glucose concentrations (Seyfried et al. 2005, Seyfried et al. 2010, Shelton et al. 2010, Seyfried 2010, Zuccoli et al. 2010). This approach would render glycolysis-dependent tumour cells vulnerable to metabolic attack. Tumour cells cannot metabolise ketone bodies for energy due to mitochondrial defects (Seyfried et al. 2010).

Apart from glucose, minimum levels of glutamine must also be maintained to fulfil in glutamatergic neurotransmission requirements, (Patel et al. 2004). The cerebral metabolic rate of glucose utilisation relative to glutamatergic neurotransmitter flux varies at a ratio of 1:1, (Patel et al. 2004). This implies that if BG levels are controlled to one-third of their safe normal values (i.e. 2 mmol/l), then the same should be done with plasma glutamine. Blood glucose levels of 2 mmol/l correspond to plasma glutamine levels of 4 mmol/l (Seyfried et al. 2010, Walsh et al. 1998). Under these conditions, the corresponding cerebral glucose concentration is 0.2 μmol/g and the cerebral glutamine concentration is 0.16 μmol/g (Shen et al. 1999).

No evidence could thus be found to refute the claim that a decrease of BG levels in the conscious brain-state to about 2 mmol/l (nearly one-third of normal values; patient-specific) would have no expected detrimental effect to normal brain cells.

How would other organs and tissues fare during periods of minimum glucose levels? PET scans reveal the uptake values of a radionuclide glucose analogue (FDG) in normal tissues and organs, as well as in their cancerous counterparts.

If the body is restricted to approximately one-third of its normal 24-hour BG concentration, say to 2 mmol/l glucose (safe for the brain, and patient-specific), normal tissue should not be compromised (cf. Table 4). Cell culture studies in fact demonstrate that tumour cells bearing cancers' metabolic changes are uniquely sensitive to inhibition of glycolysis, unlike their normal cell counterparts (Gatenby et al. 2004). This suggests a potential therapeutic window.

If a patient is subjected to a high-ketogenic diet, which would allow the brain to adapt to ketone bodies rather than glucose as its primary energy source, then plasma glucose concentrations could probably be lowered to below 2 mmol/l, say to 1 mmol/l or lower, without adversely affecting the brain.

TABLE 4
FDG uptake classification in normal tissues (PET-derived data).

| Tissue/Organ | SUV ± σ | Ref. |
|---|---|---|
| Cerebellum | 8.22 ± 2.40 | Wang et al. 2007 |
| Palatine tonsils | 4.08 ± 1.51 | Wang et al. 2007 |
| Tongue | 1.60 ± 0.83 | Wang et al. 2007 |
| Thyroid gland | 1.45 ± 0.57 | Wang et al. 2007 |
| Oesophagus | 1.61 ± 0.61 | Wang et al. 2007 |
| Breast | 0.57 ± 0.32 | Wang et al. 2007 |
| Myocardium | 4.33 ± 4.18 | Wang et al. 2007 |
| Liver | 2.06 ± 0.45 | Wang et al. 2007 |
| Pancreas | 1.48 ± 0.33 | Wang et al. 2007 |
| Upper stomach | 2.33 ± 1.1 | Wang et al. 2007 |
| Ascending colon | 1.25 ± 0.63 | Wang et al. 2007 |
| Rectum | 1.58 ± 0.79 | Wang et al. 2007 |
| Prostate | 1.90 ± 0.37 | Wang et al. 2007 |
| Testes | 2.73 ± 0.60 | Wang et al. 2007 |
| Lung | 0.64 ± 0.20 | Wang et al. 2007 |
| Skeletal muscle | 0.77 ± 0.15 | Van Loon et al. 2001 |

SUV = mean Standardised Uptake Value; σ = standard deviation

### Glucose (and Glutamine) Deprivation as an Adjuvant Metabolic Therapeutic for High-Glycolytic Cancers

The complete eradication of tumour cells is often unfeasible, particularly in solid high-glycolytic tumours of internal organs (Gatenby 2009). According to a recent paper of Gatenby (2009), attempting to kill tumour cells altogether might actually strengthen and aid therapy-resistant cells to flourish. Also, excessive cytotoxic therapy may lead to the pruning of too many tumour vessels, which compromises the delivery of cytotoxic therapies and causes hypoxia (Jain 2009), thus leading to more aggressive and highly glycolytic cells.

Cancer control seems to be a more viable goal than the full cure of cancer (with the latter implying eradication of all cancer cells), (Mathews et al. 2010, Gatenby 2009). Considering Gatenby's recent insights, an ideal control regime is hypothesized in FIG. 2. It will also later become clear that the hypothesised therapies will not prune tumour vessels with the associated problems as discussed by Gatenby (2009).

Insulin potentiation treatment (IPT), (Ayre et al. 1986) generally represents the antithesis of the sought anti-tumour effects. IPT reduces blood glucose concentration by storing BG (also in cancer cells). These cells may have a two- to 10-fold insulin sensitivity compared to normal cells (Pollak 2009, Evans et al. 2005, Ilvesmaki et al. 1993), leading to relatively much larger quantities of BG being stored in cancer cells than in normal cells. The tipping point where IPT may help, to where it will actually accelerate tumour growth, is never known (Gerstein 2010), which makes it a potentially dangerous therapy.

The focus of our proposed treatments is however on reducing blood glucose (and glutamine or other metabolic energy sources) concentration by controlling the ingestion and specifically in vivo-produced glucose (and glutamine or other metabolic energy sources), leaving little blood glucose (and glutamine or other metabolic energy sources) to store or utilise, thus not needing any insulin.

The proposed deprivation of specific nutrients (such as glucose and glutamine) from the blood plasma may be achieved by extracorporeal treatment (such as haemodialysis or haemodiafiltration) of a cancer patient's blood. This implies that, among others, chemotherapeutic pharmaceutical compositions may be administered during the blood-filtering process. The extracorporeal treatment of blood could also facilitate the local treatment of rolling cancer cells by administering pro-apoptotic signals via the use of pharmaceutical compositions such as TRAIL/E-selectin (King et al. 2009, Rana et al. 2008).

The applicants are aware of the following US patent documents pertaining to the metabolic treatment of cancers, or to novel haemodialysis or haemodiafiltration installations:
Chemoradiotherapeutic approaches, which are directed at either influencing metabolic pathways, or are directed at metabolic enzymes, to exploit the bio-energetics of tumours: 2010/0099726; 2010/0075947; 2008/0063637; 2010/0197612; 2005/0214268; 2003/0069200; 2010/0014637; U.S. Pat. No. 4,303,636; 2003/0228568; 2006/0128777; 2008/0319054; U.S. Pat. No. 5,069,662; 2003/0125283; 2002/0193313; U.S. Pat. No. 7,582,619; 2009/0226427.
Extracorporeal treatment of blood, which is directed at depriving blood of certain nutrients, or at introducing certain compounds to the blood, based on haemodialysis or haemodiafiltration. Extracorporeal control of blood glucose involve the use of insulin, in all cases: U.S. Pat. No. 5,851,958; U.S. Pat. No. 4,370,983; U.S. Pat. No. 7,338,461; U.S. Pat. No. 3,946,731; 2010/0266589; 2010/0143324; U.S. Pat. No. 5,646,185; 2009/0169591; 2005/0208023; 2003/0113746; 2005/0136502; 2003/0017995; 2006/0035844; 2003/0045582; 2007/0021357; U.S. Pat. No. 4,861,485; U.S. Pat. No. 7,758,533; 2010/0114002; 2007/0135750; 2005/0274672; 2001/0039392; 2009/0139930; U.S. Pat. No. 5,277,820.
Novel apparatus for performing haemodialysis or haemodiafiltration: U.S. Pat. No. 7,604,739; 2010/0111908; U.S. Pat. No. 4,702,829; U.S. Pat. No. 6,610,027; U.S. Pat. No. 5,518,623; U.S. Pat. No. 3,441,136; U.S. Pat. No. 5,536,412.

No existing patent could be found which do/does any of the following:
the use of extracorporeal treatment of a cancer patient's blood, for the removal of blood glucose to minimum-acceptable levels, without the use of pharmaceutical compositions such as insulin.
the use of the extracorporeal treatment of a patient's blood, for the removal of blood glutamine to minimum acceptable levels, without the use of pharmaceutical compositions such as phenylacetate.
employ a TRAIL/E-selectin coated dialysis membrane, to facilitate local treatment of rolling cancer cells.
use pharmaceutical compositions or stress modulators (e.g. benzodiazepines such as midozalam) to suppress brain blood-glucose demand in combination with extracorporeal treatment of a cancer patient's blood.
use pharmaceutical compositions (e.g., biguanides such as metformin) to suppress hepatic glucose supply, in combination with extracorporeal treatment of a cancer patient's blood.
use a computer-controlled system as add-on to a conventional dialysis machine, for the purpose of treating patients with cancerous or non-cancerous proliferative disorders by withdrawing glucose (and glutamine or other metabolic energy sources) from the patient's blood to force high-glycolytic proliferative disorders into apoptosis or necrosis, whilst simultaneous electroencephalography (EEG) monitoring provides feedback to the computer controller to ensure normal functioning of the patient's brain.
the extracorporeal treatment of the blood of a patient suffering from non-cancerous proliferative disorders, specifically by depriving the patient's blood from the main metabolic energy sources, namely glucose and glutamine, in combination with a plurality of nutrients, hormones, or pharmaceutical compositions.

### SUMMARY

According to at least one disclosed embodiment, there is provided a haemodialysis machine retrofit and control installation, which includes an intake-flow blood glucose sensor, connectable to the blood intake-flow of the haemodialysis machine; an intake-flow blood glutamine sensor, connectable to the blood intake-flow of the haemodialysis machine; a return-flow blood glucose sensor, connectable to the blood return-flow of the haemodialysis machine; a return-flow blood glutamine sensor, connectable to the blood return-flow of the haemodialysis machine; a dialysate glucose controller for controlling the glucose concentrations in the dialysate; a dialysate glutamine controller for controlling the glutamine concentrations in the dialysate; a central control unit, connected to the blood glucose sensors, the blood glutamine sensors, the dialysate glucose controller and to the dialysate glutamine controller for regulating the glucose and glutamine levels in the dialysate to obtain a required blood glucose concentration at the return-flow blood glucose sensor and a required blood glutamine concentration at the return-flow blood glutamine sensor; and an electroencephalograph (EEG) monitor providing the central control unit with information pertaining to spontaneous electro-cerebral activity to initiate raising of glucose and glutamine levels.

The haemodialysis machine retrofit installation may include a multi-dimensional concentration sensor for sensing any one or more of a concentration of ketone bodies, glutamine, insulin, hydrogen ions, and urea, measurement of dialysate and blood, blood conductivity, the multi-dimensional concentration sensor connectable into the blood intake flow of the haemodialysis machine, with sensor outputs connected to the central control unit.

The haemodialysis machine retrofit installation may include a pharmaceutical compound infusion module connectable into the blood return-flow of the haemodialysis machine, with its control inputs connected to an output of the central control unit.

The haemodialysis machine retrofit installation may include a dialysate glucose sensor connectable into the dialysate circuit, with sensor outputs connected to the central control unit.

The haemodialysis machine retrofit installation may include a regime database connected to the central control unit, the regime database containing treatment regimes for controlling any one or more of the dialysate glucose controller and the pharmaceutical compound infusion module. The regime database may define any one of a pre-defined metabolic energy source concentration and a predefined dose of a pharmaceutical composition for a particular condition.

Importantly the haemodialysis machine retrofit installation may include a patient monitoring unit, the patient monitoring unit operable to monitor any one or more of the following: electroencephalograph (EEG), electrocardiogram (ECG), blood glucose (body), blood glutamine (body), blood ketone (body), cerebral glucose, cerebral glutamine, cerebral ketone, blood pressure, heart rate, and blood flow rates.

According to another disclosed embodiment, there is provided a cerebral glycaemic control module, which includes an extracorporeal circulation circuit, connectable at one end to cerebral arteries and at another end to cerebral veins of a human or animal body; a blood glucose sensor and a flow rate sensor, connectable to the cerebral artery side of the circulation circuit; a blood glucose sensor and a flow rate sensor, connectable to the cerebral vein side of the circulation circuit; a blood glutamine sensor and a flow rate sensor, connectable to the cerebral artery side of the circulation circuit; a blood glutamine sensor and a flow rate sensor, connectable to the cerebral vein side of the circulation circuit; a blood ketone body sensor and a flow rate sensor, connectable to the cerebral artery side of the circulation circuit; a blood ketone sensor and a flow rate sensor, connectable to the cerebral vein side of the circulation circuit; a pharmaceutical compound infusion module disposed into the extracorporeal circulation circuit; a multi-metabolic energy source infusion module disposed into the extracorporeal circulation circuit; and a central control unit, connected to the glucose sensors, glutamine sensors, ketone body sensors, the flow rate sensors and the infusion modules, for controlling the multi-metabolic energy source infusion module.

The central control unit may be operable to dispense a pre-defined amount of a metabolic energy source into the extracorporeal circulation circuit.

The disclosed embodiments extend to a method for the extracorporeal treatment of blood to absolute minimum levels of metabolic energy sources to maintain homeostasis, the method including receiving blood from an animal or human into a haemodialysis machine retrofitted with a haemodialysis machine retrofit and control installation as described; employing on the haemodialysis machine a new pre-determined computer-controlled treatment regime for the systemic removal of metabolic energy sources to a desired level; controlling the level of metabolic energy sources in the blood over a pre-determined range by means of central control unit and retrofitted dialysis machine by optionally monitoring a patient's spontaneous electro-cerebral activity by electroencephalography (EEG) and receiving feedback to the controller to ensure spontaneous electro-cerebral activity of the patient's brain throughout the treatment; and returning the blood from the retrofitted haemodialysis machine to the animal or human.

The pre-determined treatment regime and the central control unit may define any one of a pre-defined metabolic energy source concentration and a predefined dose of a pharmaceutical composition for a particular condition.

The method may include the step of lowering concentrations of predefined metabolic energy sources in the blood through manipulation of conventional dialysis according to the pre-determined treatment regime and control to the best possible precision via the central control receiving EEG feedback of the cerebral activity of the brain, so as to protect the brain.

The method may include the step of raising concentrations of predefined metabolic energy sources in the blood through dialysis according to the pre-determined treatment regime being controlled by the central control unit, and optionally receiving EEG feedback of the cerebral activity of the brain.

The method may include the step of infusing pharmaceutical compositions into the blood according to the pre-determined treatment regime. Furthermore, the method may include the step of administering pro-apoptotic signals via the use of a dialysis membrane which is treated with a pharmaceutical composition comprised of TRAIL (Tumour Necrosis Factor (TNF) Related Apoptosing-Inducing Ligand) and E-selectin.

The method may include the step of sensing concentrations of compounds in the blood via the central control unit to direct the execution of the pre-determined treatment regime, the compounds in the blood being selected from any one or more of glucose, glutamine, and ketone bodies, and by monitoring the patient's spontaneous electro-cerebral activity by electroencephalography (EEG) and providing feedback to the controller to ensure spontaneous electro-cerebral activity of the patient's brain.

The disclosed embodiments extend also to a method of treating a proliferative disorder (cancerous or non-cancerous) in a human or animal, which includes reducing via a haemodialysis machine retrofitted with a haemodialysis machine retrofit and control installation, as described, any one or both the blood glucose concentration and the glutamine concentration in the human or animal body for a pre-defined period of time to a minimum threshold level as indicated by the onset of abolition of spontaneous electro-cerebral activity as monitored by, optionally, electro-encephalography (EEG) signals in the control program; suppressing the blood glucose counter-regulation demand mechanism in the human or animal body; and suppressing the rate of hepatic glucose production mechanism in the human or animal.

The method may include controlling the blood glucose concentration in the human or animal body to a level of optionally 2 mmol/l or lower. Also, the method may include controlling the blood glutamine concentration in the human or animal body to a level of optionally 0.3 mmol/l or lower.

Optionally, the method may include suppressing the blood glucose counter-regulation mechanism in the human or animal body by administering benzodiazepines (such as midozalam) as initiated by the central control unit. Also the method may include suppressing the hepatic glucose production mechanism in the human or animal body by administering biguanide-class pharmaceutical compositions (such as metformin) as initiated by the central control unit.

In at least one disclosed embodiment, the method may include the prior step of subjecting a human or animal body to dietary restriction of, optionally, 400 to 500 kcal per day by administering a high-ketogenic diet such as 4:1; fat: carbohydrate and protein, thus supplying the human or animal body with ketone body compounds to maintain the ketone body concentrations to, optionally, between 0.8 mmol/l and 1.6 mmol/l, controlled via the central control unit.

The method may include controlling the blood glucose concentration in the human or animal body to lower than, optionally, 2 mmol/l, whilst monitoring the patient's EEG activity and providing EEG feedback to the central control unit to control the administration of benzodiazepines, biguanides, and parenteral blood glucose infusion, to ensure spontaneous electro-cerebral activity of the patient's brain.

In addition, the method may include the local treatment of rolling cancer cells by administering pro-apoptotic signals via the use of a dialysis membrane which is treated with pharmaceutical compositions.

The disclosed embodiments extend also to a method of treating a proliferative disorder in a human or animal, which includes isolating the blood circulation system in any one of a limb and an organ of a human or animal body; and reducing extracorporeally by means of a haemodialysis machine retrofitted with a haemodialysis machine retrofit and control installation as described, any one or both of the blood glucose concentration and the blood glutamine concentration in any one of the isolated limb and the organ to a blood glucose level of lower than, optionally, 0.1 mmol/l and to a blood glutamine level of lower than, optionally, 0.3 mmol/l for a pre-defined period of time.

The disclosed embodiments extend also to a method of treating a proliferative disorder in a human or animal, which includes isolating the cerebral circulation system of a human or animal body from the rest of the blood circulation system; controlling extracorporeally the glucose concentration in the cerebral circulation system via a new control system to a normal level of, optionally, between 0.2 μmol/g to 0.4 μmol/g for a pre-defined period of time still ensuring spontaneous electro-cerebral activity as monitored by, optionally, electroencephalography (EEG); and controlling the glutamine concentration in the cerebral circulation system to a normal level of, optionally, between 0.1 μmol/g to 0.3 μmol/g for a pre-defined period of time to maintain spontaneous electro-cerebral activity as monitored by, optionally, electroencephalography (EEG)

The method may include reducing extracorporeally the blood glucose concentration in the human or animal body by means of a haemodialysis machine retrofitted with a haemodialysis machine retrofit and control installation as described to, optionally, between 0.8 mmol/l and 0.1 mmol/l for a pre-defined period of time and suppressing the rate of hepatic glucose production in the human or animal body.

The method may include suppressing any one of the rate of glucose demand from the rest of the blood circulation system of the body and of hepatic glucose production in the human or animal body are suppressed by administering via control unit inputs benzodiazepines and biguanide-class pharmaceutical compositions. In addition, the method may include the step of administering pro-apoptotic signals via the use of a dialysis membrane which is treated with a pharmaceutical composition comprised of TRAIL (Tumour Necrosis Factor (TNF) Related Apoptosing-Inducing Ligand) and E-selectin.

### BRIEF DESCRIPTION OF THE DRAWINGS

FIG. 1 depicts the salient characteristics of a solid cancer tumour, viewed with a section of a human colon adenocarcinoma (adapted from Brahimi-Horn et al. 2007). Unlike normal tissues, cancer tumours have highly disordered, structurally defective and ineffective vascular supply (Jain 2009, Mankoff et al. 2009, Vaupel 2004). Oxygen concentration consequently decreases as the distance from capillaries increases (Jones et al. 2009, Mankoff et al. 2009, Vaupel 2004, Gray et al. 1953, Helmlinger 1997). Tumours proliferate in close proximity to the capillaries (Jain 2009, Mankoff et al. 2009), with necrosis occurring at distances further than 150 μm from the capillaries (Mankoff et al. 2009), i.e. necrosis occurs when the tumour outgrows its blood supply. A decreasing level of oxygen is accompanied by (among others) an increase in HIF-1α levels, which stimulates an increase in glucose metabolism (Vaupel 2004) and subsequent increase in inflammation and decrease in cell immune response (Jones et al. 2009, Vaupel 2004, Kroemer et al. 2008, Moreno-Sanchez et al. 2007), as well as a decrease in the extracellular pH (Mankoff et al. 2009), and an increase in the resistance to chemoradiotherapy (Vaupel 2004, Gray et al. 1953, Moreno-Sanchez et al. 2007, Seyfried et al. 2010).

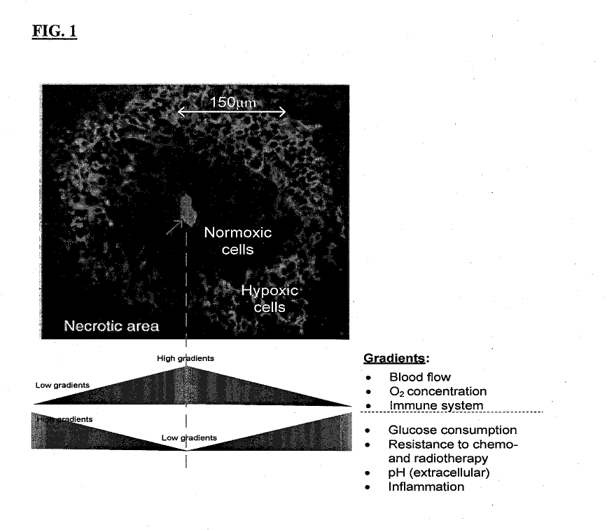

FIG. 2 illustrates a hypothetical, ideal treatment regimen for regular blood glucose-control, based on the consequential time-dependent changes in the metabolic and cell signalling modifications.

FIG. 3 gives an overview of the first installation (“External Dialysis Control Module, EDCM”) interfacing with several retrofit modules to a modern dialysis machine and patient monitors.

FIG. 4 gives an overview of the second installation (“Cerebral Glycaemic Control Module, CGCM”) interfacing with several retrofit modules to a modern dialysis machine and patient monitors.

As those in the art will appreciate, the following description describes certain disclosed embodiments in detail, and is thus only representative and does not depict the actual scope of the invention. Before describing the disclosed embodiments in detail, it is understood that the invention is not limited to the particular methodologies, systems, and molecules described, as these may vary. It is also to be understood that the terminology used herein is for the purpose of describing particular disclosed embodiments only, and is not intended to limit the scope of the invention defined by the appended claims.

### DETAILED DESCRIPTION

The disclosed embodiments provide methods and describes installations for treating proliferative disorders in a patient, by extracorporeal treatment of the patient's blood. The proliferative disorders may be of the cancerous or non-cancerous types. This detailed description will, however, only describe the treatment of cancerous proliferative disorders, as both these and non-cancerous proliferative disorders make use of, but are not limited to, glucose (and glutamine) as their main metabolic energy sources.

The authors of this specification do not imply that glucose (or glutamine and other metabolic energy sources) causes cancer. Many genetic and environmental factors influence the risk for developing cancer. Glucose is however the primary energy source for high-metabolic cancer cells, with glutamine being the secondary critical energy source for malignant cells. Adequate availability of glucose (and glutamine and other metabolic energy sources) therefore creates a suitable breeding ground for the progression of cancer.

Based on this knowledge, the disclosed embodiments propose an installation to reduce primarily the glucose (and secondarily, glutamine or other metabolic energy sources) supply needed by proliferative cells. The installation may however also enrich or deprive the patient's blood plasma with/of components such as hormones and pharmaceutical compositions.

The distinctive feature that will be focussed on by this installation and method of treatment is the fact that most types of cancer (and other proliferative) tissue require a far greater supply of glucose than non-cancerous (or non-proliferative) tissue. This is due to the high-glycolytic turnover in cancer (and other proliferative) cells.

A careful balancing act is therefore required to provide non-cancerous (or normal) cells with the minimum required glucose supply, whilst simultaneously limiting the glucose supply to cancerous (and other proliferative) cells to such an extent that cancer (or other proliferative) cells are forced into necrosis or apoptosis. This change in the glucose extra-cellular environment should optionally be sudden to prevent the cancer (or other proliferative) cells from converting to an alternative metabolic energy pathway, such as the fatty acid pathway.

A critical concern associated with the disclosed embodiments and method of treatment is the risk of brain damage associated with severe hypoglycaemia. The disclosed embodiments lower blood glucose levels, into the severe hypoglycaemic range, so as to reduce the glucose available to high-metabolic cancer (and other proliferative) cells to sub-critical levels. This requires precision control of the blood glucose (BG) concentrations by, among others, monitoring the patient's electroencephalogram (EEG) status, which provides the central control unit with information pertaining to spontaneous electro-cerebral activity, thus facilitating haemodialysis treatment without adversely affecting cerebral function.

By subjecting a patient to a high-ketogenic diet for a suitable period of time prior to the proposed treatment, the brain would adopt such ketone bodies as its primary energy source, rather than glucose (Seyfried 2010, Seyfried et al. 2010). This implies that plasma glucose concentrations could then probably be lowered to below 2 mmol/l, say to 1 mmol/l or lower, without adversely affecting the brain. The glucose concentrations correspond to 1.6 mmol/l and 0.8 mmol/l, respectively (Seyfried et al. 2010).

The disclosed embodiments and method of treatment are concerned with the deprivation of glucose and glutamine (among other nutrients and metabolic energy sources) from the blood plasma, and with the enrichment of the blood plasma by, among others, chemotherapeutic pharmaceutical compositions (such as TRAIL/E-selectin, cisplatin), anxiolytics (such as midozalam), or hepatic glucose suppressors (such as the biguanide-class pharmaceutical, metformin).

Insulin administration is the conventional way to reduce blood glucose levels. Insulin allows glucose to be stored in glycogen stores (viz. glycogenesis), but also allows cells to accept glucose molecules into the cells for their metabolic energy requirements. Insulin thus also helps to very efficiently accept and store glucose in the much more insulin-sensitive cancer (and other proliferative) cells, compared to normal cells.

The efficacy of insulin potentiation will therefore be strongly dependent on the metabolic activity of the targeted cancer (or proliferative disorder) and on the size of the tumour relative to that of normal tissue. It is difficult to correctly establish these variables and their interrelationships. The main purpose of the disclosed embodiments and method of treatment is the much safer option where glucose is restricted from cancerous or non-cancerous proliferative cells.

One way of removing molecules from blood is by means of “dialysis”, also known as “extracorporeal treatment of blood” (U.S. Pat. No. 5,851,985; U.S. Pat. No. 4,191,182; U.S. Pat. No. 3,946,731, US 2010/0266589; US 2010/0143324, U.S. Pat. No. 5,646,185). The dialysis process can be customized to remove certain molecules from the blood by selecting the correct dialysis process, semi-permeable membrane, and dialysate solution.

Glycaemic homeostasis in the body is achieved by secreting insulin when blood glucose levels rise above normal levels. When blood glucose levels fall below normal levels, the blood glucose counter regulation system (BGCRS) increases blood glucose levels by converting stored glycogen to glucose (viz. glucogenolysis) or converting lactate, glycerol, and glucogenic amino acids to glucose (viz. gluconeogenesis).

Hormones such as glucagon, cortisol, epinephrine and adrenalin trigger these glucose up-regulations. Stress hormones such as cortisol also trigger the up-regulation of blood glucose in response to psychological and/or physiological stress. This up-regulation however, does not necessarily accompany hyperglycaemia, as the additional blood glucose supply is intended for coping with stress.

In order to take extensive control of a patient's blood glucose concentration, the blood glucose counter-regulation system (i.e., “demand and supply”) should be suppressed by means of pharmaceutical composition administration. Biguanides (such as metformin) are well-known diabetic pharmaceutical compositions used for inhibiting hepatic glucose synthesis (i.e., “supply”). Anxiolytics (such as midozalam) might also be administered to the patient to reduce the up-regulation (i.e., “demand”) caused by psychological and/or physiological stress.

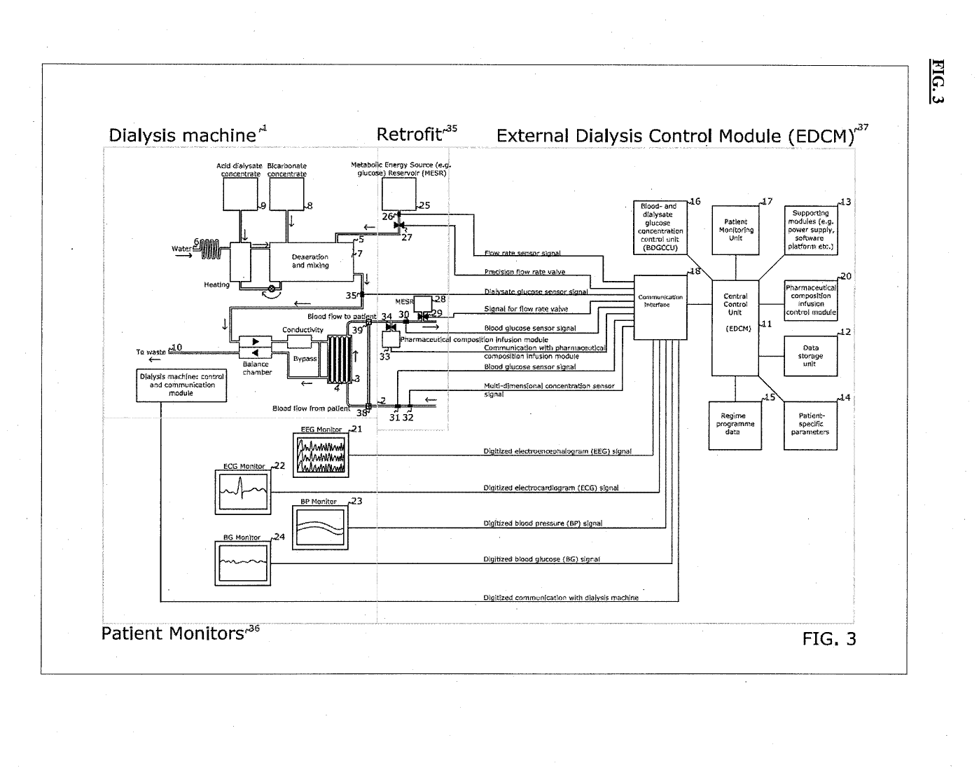

The inventive installation communicates with a selected range of conventional dialysis machines, cf. FIG. 3. None of the inherent technologies included in any dialysis machine is being claimed in this patent specification. Item 1 in FIG. 3 encapsulates the components of a typical dialysis machine Item 35 displays the required retrofit units. Item 37 describes the inventive External Dialysis Control Module (ECDM).

New therapies have recently been investigated for treating rolling cancer cells, including leukemic and metastatic cells (King et al. 2009, Rana et al. 2008). With a 1-hour rolling exposure to a functionalized TRAIL (Tumour Necrosis Factor (TNF) Related Apoptosing-Inducing Ligand) and E-selectin surface, up to 30% of cancer cells received an apoptopic signal and were killed (Rana et al. 2008). TRAIL however also exerts its cytotoxic effects on normal liver and brain cells (Rana et al. 2008). A localised TRAIL delivery system is therefore warranted.

Haemodialysis presents a continuous (and localised) filtering of the blood. The merger of TRAIL/E-selectin-based therapy with haemodialysis therefore presents a unique opportunity to achieve killing of leukemic or metastatic cells. This further raises the possibility to perform TRAIL-haemodiafiltration during cancer surgery to “filter” out any leaking cancer cells.

An installation is proposed to control the blood glucose (and glutamine or other metabolic energy source) levels of a patient suffering from cancerous or non-cancerous proliferative disorders (cf. FIG. 3), by controlling a typical modern dialysis machine 1 and providing complementary retrofitted hardware modules 35 to aid with the required precision control. The installation is an essential technology used in a proposed method for the treatment of cancerous or non-cancerous proliferative disorders where the blood glucose level (and levels of glutamine and other metabolic energy sources) of a patient is controlled to a very low glycaemic level (and a low glutamatergic level, or low levels of other nutrients and metabolic energy sources) within a very narrow range of blood glucose (and glutamine or metabolic energy sources), by, among others, using inputs from the patient's EEG monitor.

Note that the proposed method of treatment and installation is not a conventional extracorporeal blood glucose control apparatus such as an insulin pump or artificial pancreas. Insulin will not be administered to lower blood glucose levels. Blood glucose levels will be lowered by means of supervised dialysis and not through the administration of insulin. More detail on this concept will follow later in this specification.

The external dialysis control module (EDCM) 37 is comprised of several modular units, including interfacing with the critically important EEG monitor 21. A central control unit 11 is responsible for monitoring and controlling the different modular units of the EDCM, including the EEG monitor. It is implemented as a software application executed on a personal computer-type device or proprietary-designed micro-electronic system.

The EDCM includes a data storage unit 12 where the main control unit's software application is stored. It also stores historical control data, historically measured data, reference values used for the control algorithms used by the main control system, data and parameters of different treatment regimes, communication protocols for different external modules (e.g. communication protocol for EEG machine), etc. The data storage unit may comprise a combination of random access memory (RAM) and/or mass storage devices (e.g. hard disk).

Supporting modules 13 are included to provide the EDCM installation with electrical power. These include an electrical power supply, surge protection, battery backup and isolation circuitry as required for medical instrumentation. Further supporting modules include output devices (e.g. screen/monitor, printer and speaker). Input devices are also categorised under supporting modules and include items such as a keyboard, pointing device (e.g. computer mouse, trackball, feedback from touch-screen display, etc.), buttons, levers, knobs, etc.

Communication between the different internal modules (including 11, 12, 13, 14, 15, 16, 17 and 18) of the EDCM takes place through the internal communication channels of the personal computer or proprietary electronic platform being used for the installation. If a personal computer is used, communication propagates through the internal communication hardware data busses of the system.

The EDCM installation controls the blood glucose (or glutamine and other metabolic fuels) level of the patient to a severely low blood glucose (or glutamine and other metabolic fuels) concentration within a narrow control range, by, among others, using inputs from the patient's EEG monitor. The EDCM makes use of a regime programme selected by the medical professional (user) administering the EDCM treatment. This is done by using the input devices to navigate through different treatment options displayed on the output devices.

Once a specific treatment regime has been selected, the regime programme data are retrieved from the data storage unit 12 and loaded into the regime programme data 15 module. Several options are then presented to the user (medical professional). The user selects the type of equipment and treatment specifics to be used. These parameters are then stored with the regime programme data 15.

The regime programme data 15 include a vast array of parameters relating to the treatment specifics, the dialysis machine being used, the blood glucose administration equipment being used and also the pharmaceutical composition administration equipment being used for the EDCM treatment. This module 15 may exist in the memory of the electronic platform on which the EDCM is based.

Regime programme data 15 relating to the treatment specifics include, among others, the following: the allowable range of EEG signals; the duration of the dialysis treatment; the target blood glucose (or other metabolic energy source) control level; the blood glucose (or other metabolic energy source) control range tolerated; the blood glucose (or other metabolic energy source) concentration levels when external warning alarms should be triggered; the type of pharmaceutical compositions (chemical formulations) to be administered during the treatment; and, the dosage and time-administration-profiles of the pharmaceutical compositions to be administered during the treatment.

Regime programme data 15 relating to the dialysis machine 1 include, among others, the following: the specific dialysis machine and model being used; the type of dialysis treatment to be administered (e.g. haemodialysis, haemodiafiltration, etc.); the dialysis blood flow rate; the dialysate flow rate; the time profiles for variable mixing of the dialysate (e.g. concentrations/quantities of sodium, potassium, calcium, glucose (dextrose), etc.); the specific dialyser to be used (e.g. different types of membranes have different\ clearance efficiencies); and, the communication protocol being used by the dialysis machine for external communication.

Regime programme data 15 relating to the glucose administration equipment 1 include, among others, the following: the specific glucose (or other metabolic energy source) administration device used to feed glucose (or other metabolic energy source) to the dialysate mixing chamber; the communication protocol used by the above mentioned glucose (or other metabolic energy source) administration device; the minimum allowed level of the glucose (or other metabolic energy source) administration device's glucose (or other metabolic energy source) reservoir; the specific blood glucose (or other metabolic energy source) administration device used to feed glucose (or other metabolic energy source) into an artery or vein of the patient being treated; the communication protocol used by the last mentioned blood glucose (or other metabolic energy source) administration device; and, the minimum allowed level of the last mentioned blood glucose (or other metabolic energy source) administration device's glucose (or other metabolic energy source) reservoir.

Regime programme data 15 relating to the administration of pharmaceutical compositions or chemical formulations include, among others, the type of pharmaceutical compositions or chemical formulations to be administered; and, the time-administration profiles of the different pharmaceutical compositions to be administered during the treatment.

Another level of customisation is however needed for the treatment regime. Patient-specific parameters 14 are stored in a similar way as the regime programme data 15. These parameters relate specifically to the individual to be treated. Patient-specific parameters 14 include, among others, the body characteristics of the patient such as body weight, age and gender; medical history of the patient (e.g. Type 1 diabetes, hypertension, hypotension, etc.); range of supportable blood pressure; range of supportable heart-beat rate; allergies (to prevent allergic reactions with pharmaceutical compositions or chemical formulations being administered).

The patient-specific parameters 14 and the regime programme data 15 are used in conjunction by the EDCM in the formulation of a treatment algorithm to be used and executed by the blood and dialysate glucose (or other metabolic energy source) concentration unit 16.

Communication with external equipment, including the dialysis machine, blood glucose (or other metabolic energy source) administration devices, pharmaceutical composition administration devices and patient monitors takes place via a specialised communication interface 18. The main function of the communication interface 18 is to connect, either in wired or wireless format, the external equipment being used to the EDCM.

The communication interface is responsible for the translation of data signals to and from internal and external equipment. The specific communication protocol to be used for a specific piece of external equipment is specified in the regime programme data 15.

The reference language commands for the specific equipment are stored in the data storage unit 12. Availability of a specific hardware language may be subject to a licensing agreement with the manufacturer of the specific equipment.

The communication interface 18 also includes physically compatible data ports/sockets/jacks for wired communication and the relevant RF (radio frequency) receiver/transmitter modules for communication with wireless equipment (e.g. Bluetooth or Wi-Fi). Internal communication between the internal EDCM modules and the communication interface takes place via the data buses of the electronic platform on which the EDCM is implemented.

The patient monitoring unit 17 is responsible for monitoring data received from the different patient monitors to ensure that the vital signs of the patient are within acceptable levels. It also monitors the patient's EEG status 21, as well as the various other patient monitors (22, 23, 24) to consistently check that all the equipment is working correctly and connected to the EDCM system.

Some dialysis machines incorporate patient monitors such as blood pressure and ECG monitors. For these machines, the patient monitors' data should optionally be obtained directly from the dialysis machine 1 via the communication interface 18. It is however important to ensure that EEG monitoring will also be available.

The patient monitoring unit 17 uses data from the patient-specific parameters 14 to monitor data received from patient monitors such as the EEG monitor 21, blood pressure monitor 23, ECG monitor 22, and blood glucose monitor 24. Values that are out of the tolerable ranges are immediately reported to the user (medical professional). The EDCM may respond by terminating or pausing treatment in response to alarming vital signs.

Before continuing to the description of the blood- and dialysate glucose (or other metabolic energy source) concentration control unit 16, a description is needed of the hardware modules that are used in conjunction with the dialysis machine. These modules can be retrofitted 35 to the existing dialysis machine.

The first retrofit module is a high-precision glucose (dextrose), or other metabolic energy source dispensing unit (25, 26, 27). This unit is responsible for controlled feeding of glucose (dextrose), or other metabolic energy source into the dialysate mixing chamber 5. No claims are being made to the technologies being used by any glucose (or other metabolic energy source) dispensing units. A pre-existing commercially available glucose (or other metabolic energy source) dispensing unit can be used.

The essential features of such a glucose (or other metabolic energy source) dispensing unit include:
a high-precision glucose (or other metabolic energy source) dispensing unit
digitized or electronic actuation signals, and
digitized or electronic feedback on both the glucose (or other metabolic energy source) flow rate (e.g. measured with a flow rate meter 26) and the glucose (or other metabolic energy source) reservoir's level.

The main function of the glucose (or other metabolic energy source) dispensing unit 25 is to add glucose (dextrose), or other metabolic energy source to the dialysate mix so that the dialysate concentration can be variably controlled in real-time by the blood- and dialysate glucose (or other metabolic energy source) concentration control unit 16.

The second retrofit module is the blood glucose (or other metabolic energy source) dispensing unit (28, 29). The main difference between this glucose (or other metabolic energy source) dispensing unit 28 and unit 25 is that the blood glucose (or other metabolic energy source) dispensing unit 28 dispenses glucose (or other metabolic energy source) directly to the blood to increase the systemic blood glucose (or other metabolic energy source) concentration of the patient being treated, while the glucose (or other metabolic energy source) dispensing unit 25 dispenses glucose (or other metabolic energy source) into the dialysate mixing chamber 5 to increase the glucose (or other metabolic energy source) concentration of the dialysate.

The blood glucose (or other metabolic energy source) dispensing unit 28 requires similar essential features as listed earlier for the high-precision glucose (or other metabolic energy source) dispensing unit 25.

A third retrofit module is the pharmaceutical composition infusion module 33 administering pharmaceutical compositions into the systemic blood stream of the patient. Again, no claims are being made toward the existing technologies being used by such a device. The pharmaceutical composition infusion module 33 should optionally be able to administer multiple pharmaceutical compositions or chemical formulations at precise infusion rates as instructed by the EDCM's pharmaceutical composition infusion control module.

Pharmaceutical compositions or chemical formulations to be administered by the infusion module 33 include suppressors of the hepatic glucose production system (e.g. the biguanide-class pharmaceutical composition, metformin) and also anxiolytics to suppress the blood glucose counter regulation system (e.g. the benzodiazepine-class pharmaceutical composition midazolam; or β-adrenergic blockers such as propranolol), anti-coagulants (e.g. heparin), and prostacyclins such as flolan.

A fourth retrofit module required is a multi-dimensional concentration sensor 32. This module comprises an array of sensors measuring concentrations of different substances or blood parameters such as levels of ketone bodies, amino acids, acidity, alkalinity, viscosity, etc. These sensors may provide constant or regular digitized or electronic feedback signals to the EDCM via the EDCM's communication interface 18.

A fifth and very important retrofit module comprises the blood glucose (or other metabolic energy source) monitors 30 and 31. Blood glucose (or other metabolic energy source) monitors 30 and 31 measure blood glucose (or other metabolic energy source) flowing to and from the systemic blood glucose (or other metabolic energy source) circulation circuit. The actual amount of glucose (or other metabolic energy source) removed from the blood can therefore be approximated by using the difference in the two blood glucose (or other metabolic energy source) concentrations and the blood flow rate. Again, these sensors may provide digitized or electronic feedback signals to the EDCM via the communication interface 18.

The blood glucose (or other metabolic energy source) sensors are not adversely affected by their plasma environment, as plasma water is free from contaminants such as proteins, albumin, and white and red blood cells, which could affect the operation of the sensor.

Returning to the internal EDCM modules, the blood- and dialysate glucose concentration control unit (BDGCCU) 16 is a digital control system. Its main function is to control the dialysis machine 1 and retrofit modules 35 to precisely control the blood glucose (or other metabolic energy source) level of the patient being treated.

The BDGCCU control algorithm uses several inputs and includes, among others, the following:
Blood glucose (or other metabolic energy source) level of systematic circulation circuit measured by blood glucose (or other metabolic energy source) monitors 31, 24.
Blood glucose (or other metabolic energy source) level of dialysed blood returning to the systematic blood circulation circuit measured by blood glucose (or other metabolic energy source) monitor 30.
Dialysate glucose concentration measured by glucose sensor 35.
Multiple parameters such as acidity, ketone concentration, glutamine concentration, etc., measured by the multi-dimensional concentration sensor 32.
Feedback from patient monitors 36 including EEG (electroencephalograph) monitor 21, ECG (electrocardiograph) monitor 22 and BP (blood pressure) monitor 23.
Blood clearance rates obtained from the dialysis machine 1.

The BDGCCU also uses inputs from equipment being actuated to ensure that control actions are performed as commanded. These inputs include, among others, the following:
Flow rate 26 of glucose (or other metabolic energy source) administration module 25.
Flow rate 29 of blood glucose (or other metabolic energy source) administration module 28.
Status info received from the dialysis machine 1.
Status feedback from internal and external modules comprising the EDCM, being controlled or being communicated with by the EDCM.
Communication status feedback from the communication interface 18 to ensure that all devices linked with the EDCM are working properly.

The BDGCCU control philosophy is as follows:
The desired blood glucose (or other metabolic energy source) control set-point is obtained from the regime programmed data 15.
The dialysis process is initiated by the BDGCCU, 16.
The amount of glucose (or other metabolic energy source) removed from the blood is closely monitored by calculating the glucose clearance rate by using the difference in blood glucose (or other metabolic energy source) concentrations of blood to and from the dialysis machine, as well as the blood flow rate.
The blood glucose (or other metabolic energy source) level as measured by glucose (or other metabolic energy source) monitors 24 and 31 and by EEG monitor 22 is continuously monitored to adjust control actions within the safety bounds of the dialysis process.
The blood glucose (or other metabolic energy source) level is precisely controlled within a very narrow control range.
A method of preventing blood glucose (or other metabolic energy source) levels from falling below the control range threshold is to adjust the glucose (or other metabolic energy source) concentration of the dialysate dynamically by actuating infusion module 25.
The blood glucose (or other metabolic energy source) level can however be increased immediately by infusion module 28 when the blood glucose (or other metabolic energy source) level approaches the lower control range threshold.
The administration regime for pharmaceutical compositions or chemical formulations to be administered by the infusion module 33 is stored in the regime programme data 15. The BDGCCU (16) executes the regime by sending actuation signals to the infusion module.
The duration of the dialysis session is obtained from the regime programme data 15.
Patient monitors 36 are continuously monitored to ensure vital signs remain within the defined limits as specified by the regime programme data 15. In reaction to this monitoring, the dialysis process may be paused, flow rates adjusted, or a warning alarm created to inform the user (medical professional) of any potential adverse event.
Patient EEG signals 22 are continuously monitored to ensure that there are no rapid changes in the brain's glucose environment.
All control actions and monitored events are stored in the data storage unit 12.

The above-described method and installation refer to the patient's body and brain receiving the same extracorporeal blood treatment.

In yet another disclosed embodiment, it is possible to control the cerebral glucose environment of the patient independently of the blood glucose environment in the rest of the patient's body. Therefore the blood glucose level of the patient can now be divided into two separate zones, the cerebral zone and the body zone.

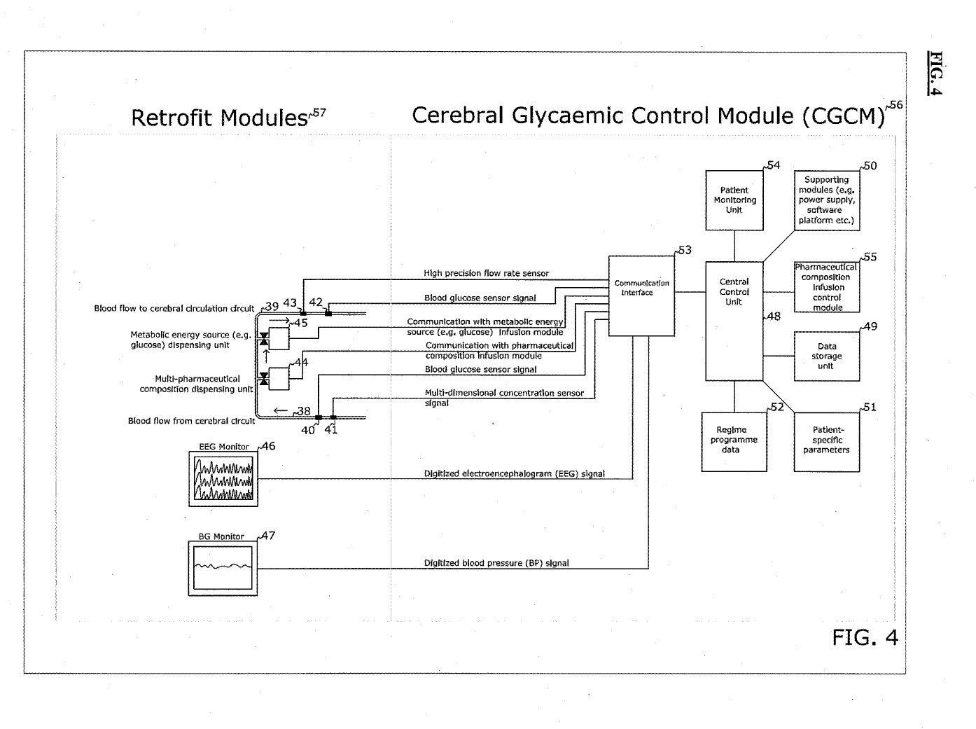

The body zone will be treated as explained in the previous disclosed embodiment by using the External Dialysis Control Module (EDCM), and as described in FIG. 3. The cerebral zone will however be treated without dialysis, and by employing a Cerebral Glycaemic Control Module (CGCM), as described in FIG. 4. The installation is similar to that in the previous disclosed embodiment. However, the previously used external dialysis control module (EDCM) 37 is now termed the cerebral glycaemic control module (CGCM) 56. The patient's spontaneous electro-cerebral activity could be monitored by EEG (46) signalling to the central control unit, thus facilitating haemodialysis treatment without adversely affecting cerebral function.

The CGCM 56 is comprised of several modular units, cf. FIG. 4. A central control unit 48 is responsible for monitoring and controlling the different modular units of the CGCM. It is implemented as a software application executed on a personal computer-type device or proprietary-designed micro-electronic system.

The CGCM includes a data storage unit 49 with the same features as the EDCM's data storage unit 12. Supporting modules 50 are similar to those of 13. Communication between the different internal modules (including 48, 49, 50, 51, 52, 53, 54 and 55) of the CGCM are similar to those of the EDCM (11, 12, 13, 14, 15, 16, 17).

The CGCM installation controls the cerebral glucose level of the patient to a specified set-point, within a narrow control range to ensure the brain does not enter a hypoglycaemic state. The CGCM makes use of a regime programme selected by the medical professional (user) administering the CGCM treatment. This is done by using the input devices, which include allowable EEG signal ranges, to navigate through different treatment options displayed on the output devices.

Once a specific treatment regime has been selected, the regime programme data are retrieved from the data storage unit 49 and loaded into the regime programme data 52 module. Several options are then presented to the user (medical professional). The user selects the type of equipment and treatment specifics to be used. These parameters are then stored with the regime programme data 52.

The regime programme data 52 include a vast array of parameters relating to the treatment specifics, the patient monitors being used (which may include EEG monitoring), and the blood glucose administration equipment being used and also the pharmaceutical composition administration equipment being used for the CGCM treatment. This module 52 may exist in the memory of the electronic platform on which the CGCM is based.

Regime programme data 52 relating to the treatment specifics include, among others, the following: The allowable ranges of EEG signals; the duration of the control; the target cerebral glucose control level; the cerebral glucose control range tolerated; the cerebral glucose concentration levels when external warning alarms should be triggered; the type of pharmaceutical compositions (chemical formulations) to be administered during the treatment; and, the dosage, dosage functions and/or time-administration-profiles of the pharmaceutical compositions to be administered during the treatment.

Regime programme data 52 relating to the glucose administration equipment 44 include, among others, the following:
the specific blood glucose administration device used to feed glucose into the cerebral circulatory network of the patient being treated,
the communication protocol used by the blood glucose administration device, and
the minimum allowed level of the blood glucose administration device's glucose reservoir.

Patient-specific parameters 51 and regime programme data 52 similar to those of the EDCM's 14 and 15, respectively.

The patient-specific parameters 51 and the regime programme data 52 are used in conjunction by the CGCM to control the blood glucose level in the patient's brain.

Communication with external equipment, blood glucose administration devices, pharmaceutical composition administration devices and patient monitors takes place via a specialised communication interface 53, similar to that of the ECM's communication interface 18.

Before continuing to the description of the CGCM's control unit 48, a description is needed of the hardware modules that are used in conjunction the proposed installation. These modules can be retrofitted 35.

The first retrofit module is the glucose dispensing unit 44. No claims are being made towards the existing technologies being used by such a device. This unit precisely dispenses glucose into the cerebral circulation network. The exact rate of glucose dispensing is determined by the CGCM's control unit 48. The essential features of the glucose dispensing unit are the same as those of the EDCM (25, 26, 27).

The second retrofit module is the multi-pharmaceutical composition infusion module 45, and operates identically to that of the EDCM (33).

A third retrofit module required is a multi-dimensional concentration sensor 41. This module comprises an array of sensors measuring concentrations of different substances or blood parameters such as ketone levels, amino acids, acidity, alkalinity, viscosity, etc. These sensors may provide constant or regular digitized or electronic feedback signals to the CGCM via the CGCM's communication interface 55.

A fourth and very important retrofit module comprises the blood glucose monitors 40 and 42. Blood glucose monitors 40 and 42 measure blood glucose flowing to and from the cerebral circulation network. Again, these sensors may provide digitized or electronic feedback signals to the CGCM via the communication interface 55.

Returning to the internal CGCM modules, the CGCM's control system is a digital control system. Its main function is to control the cerebral glycaemic environment by controlling and communicating with retrofit modules 40, 41, 42, 43, 44 and 45 to precisely control the blood glucose level of the patient's brain.

The control algorithm uses several inputs and includes, among others, the following:
Blood glucose level of blood flowing from the cerebral blood circuit, prior to possible glucose administration by 44, measured by blood glucose monitors 40.
Blood glucose level of blood flowing to the cerebral blood circuit, after possible glucose administration by 44, measured by blood glucose monitors 42.
Blood glucose (or other metabolic energy source) level of blood in the systemic circulation network of the patient measured by blood glucose (or other metabolic energy source) monitor 47.
Multiple parameters such as acidity, ketone concentration, glutamine concentration, etc., measured by the multi-dimensional concentration sensor 41.
Precise blood flow rate through the retrofit circuit 57.
Feedback from patient monitors (e.g. EEG monitor 46, systemic blood glucose or other metabolic energy source control monitor 47).

The control system also uses inputs from equipment being actuated to ensure that control actions are performed as commanded. These inputs include, among others, the following:
Flow rate of the glucose (or other metabolic energy source) administration module 44.
Status feedback from internal and external modules comprising the CGCM, being controlled or being communicated with by the CGCM.
Communication status feedback from the communication interface 55 to ensure that all devices linked with the CGCM are working properly.

The control philosophy works as follows:
The desired blood glucose (or other metabolic energy source) control set-point is obtained from the regime programmed data 52.
The cerebral blood glucose level as measured directly by glucose monitors 40 and 42 and indirectly by the EEG monitor 46, is continuously monitored to adjust control actions.
The cerebral glucose level is precisely controlled within a very narrow control range.
Glucose administration is limited in such a way to provide only the required glucose to maintain the cerebral glucose environment in a normoglycaemic state. Care is taken to limit excess cerebral glucose from elevating the glucose level of the systemic circulation network.
The administration regime for pharmaceutical compositions or chemical formulations to be administered by the infusion module 45 is stored in the regime programme data 52. The CGCM 48 executes the regime by sending actuation signals to the infusion module.
Patient monitors are continuously monitored to ensure vital signs remain within the defined limits as specified by the regime programme data 52.
Patient EEG signals are also continuously monitored to ensure that there are no rapid changes in spontaneous electro-cerebral activity.
All control actions and monitored events are stored in the data storage unit 49.

The proposed treatment using the proposed installations is to be conducted under the close and continuous supervision of a medical professional. The CGCM 56 discriminates between minor difficulties or problems that can be solved automatically and more serious problems which require the attention of a medical professional.

A first dialysis treatment (by removing glucose and glutamine to minimum-acceptable levels) should typically last for several hours. The treatment may require regular follow-up treatment, to ensure control of proliferative cells (cancerous or non-cancerous types). Repeat treatments may be delayed as proliferative cell (cancerous or non-cancerous types) growth becomes controlled. Repeating these therapeutic sessions should not pose risks, due to only minimal side-effects that are expected. It is also highly unlikely that the treated proliferative disorder will have time to develop resistance to this rapidly-deployed metabolic treatment.

### Disclosed Embodiments

In at least one disclosed embodiment of the proposed treatment and installation, ill-defined and difficult-to-reach cancerous or non-cancerous, highly glycolytic proliferative disorders, including metastatic ones, may be treated. Therefore, high-flux haemodiafiltration may potentially be used to control blood glucose levels (and levels of glutamine and other metabolic energy sources) in the body as a whole, viz. supply-side blood-glucose energy management.

Dialysis may be applied via the brachial artery (Daugirdas et al. 2001, Bosch et al. 1993, Levy et al. 2009). Controlling the expected flow of inflammatory chemicals from the resulting necrotic material (Wheatley et al. 2005) will be accounted for by the removal of dialysis waste products.

Cytotoxic pharmaceutical compositions (such as cisplatin) may additionally be administered with this treatment, but will in this case still have systemic effects due to, among others, the highly glycolytic brain also receiving the pharmaceutical composition.

The minimum blood glucose energy demand from the brain can be lowered further (below 2 mmol/l; patient specific), by administering an appropriate stress modulator (such as benzodiazepines or β-adrenergic blockers), (Siegel et al. 1998). If blood glucose concentration falls below required levels, as, among others, shown by EEG signals, it can be corrected externally by parenteral administration of glucose to the replacement fluids being mixed with dialysed blood before returning to the body (Levy et al. 2009).

By placing the patient on a high-ketogenic diet for several days before the treatment, it should be possible to treat the patient at even lower blood glucose concentrations than 2 mmol/l, say 1 mmol/l or lower. This is facilitated by the body adapting to ketone bodies as energy source, rather than glucose. The plasma concentration of ketone bodies should be regulated to between 1.6 and 0.8 mmol/l under these conditions (Seyfried et al. 2010). Normal cells, including brain tissue can adapt to this new energy source, whilst cancer cells cannot adapt (Seyfried et al. 2010, Zuccoli et al. 2010). To ensure safe treatment, EEG signals should be closely monitored.

Glutamine (or other metabolic energy sources) could also be removed by haemodialysis. However, the glutamatergic neurotransmission requirements must now be considered. With blood glucose levels of 1.5 to 2 mmol/l (i.e. nearly one-third of normal value; patient-specific), plasma glutamine levels of 0.3 to 0.45 mmol/l should be maintained (Kaadige et al. 2009). Similar plasma glutamine to blood glucose ratios should be maintained beyond the above levels, due to the linear correlation that exists between these two quantities.

Cahill et al. (1966) shows that in the presence of ketone bodies (amount not specified), the brain glucose levels can be lowered to 8 mg/dl (equivalent to 0.45 mmol/l). No cognitive abnormalities were noted. It however remains contentious as to the required period for the brain to convert from glucose utilisation to that of ketone bodies.

While it is anticipated that some cancerous or non-cancerous proliferative cells will survive due to the fact that it is not possible to extract all blood glucose and glutamine (or other metabolic energy sources) in vivo without doing some damage to normal cells (Kitano 2004, De Berardinis et al. 2010, Cahill 2006) and especially the brain, it is possible that a significant percentage of cancer cells will not survive the restricted metabolic conditions (Mathews et al. 2010).

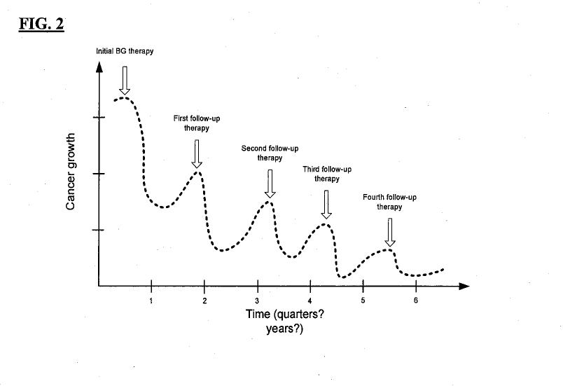

Due to the anticipated survival of some cancerous or non-cancerous proliferative cells, it is envisaged that the BG-control haemodialysis should be required to be repeated, cf. FIG. 2, and would be patient-specific. The described haemodiafiltration treatment affords low risk, based on it being a mature clinical procedure for kidney disease. It may even be home-administered in some cases once the full procedure has matured for therapy of proliferative disorders.

In another disclosed embodiment of the proposed treatment and installation, the dialysis of glucose (and glutamine or other metabolic energy sources) may be applied to an isolated limb instead of to the whole body. If a tumour is well localised in a limb (or in an organ such as the liver), without metastasis, one may control the blood glucose (and glutamine or other metabolic energy sources) in that area alone through haemodialysis.

Isolation of limbs as a cancer-fighting strategy is already well-established (Eggermont et al. 2003, Bonvalot et al. 2009, Grünhagen et al. 2005). As an extension of this therapy, the isolated limb with cancerous or non-cancerous tumour could be haemodialysed specifically of glucose and glutamine (Daugirdas et al. 2001, Bosch et al. 1993, Levy et al. 2009), or of other metabolic energy sources, by customizing the dialysate solution (Bosch et al. 1993).

Furthermore, the beneficial acidic extra-cellular environment of a cancer tumour may be neutralised by using bicarbonate, which would inhibit glycolytic energy production in the short term (Tennant et al. 2010). In the longer term it should inhibit tumour cell invasion (Tennant et al. 2010).

Resting skeletal muscle rely mainly (approximately 60%) on plasma fatty acids for energy source supply, with the balance being provided by blood glucose (Moreno-Sanchez et al. 2007). Furthermore, normal cells are more metabolically adaptable than their cancerous counterparts with their glycolytic phenotypes (Kitano 2004, Seyfried et al. 2010, Gatenby et al. 2004). The surrounding normal cells of the isolated limb should therefore be able to utilise other oxidisable substrates, such as fatty acids, when deprived of glucose (Kitano 2004, Gatenby et al. 2004).

Blood glucose levels can now also be decreased to much lower than 2 mmol/l (patient-specific), as the brain's blood supply is unaffected. Table 4 shows that muscles typically have SUV values of around 0.77, which indicates small blood glucose need. This SUV value corresponds to glucose utilization of 0.1 mmol/l, (Wahren et al. 1971, Wahren et al. 1978). From Table 2, it may follow that solid cancer tumour cells, with their much higher SUV values (thus much higher blood glucose need), could potentially be dealt a severe metabolic blow when blood glucose levels approximate 0.1 mmol/l by extracting glucose via haemodiafiltration.

With reference to the in vitro normoxic results in Table 3, it is hypothesized that, depending on the treatment blood glucose control levels, in vivo hypoxic cancers (and other non-cancerous proliferative disorders) may possibly be controlled (not necessarily fully eradicated) with dialyses periods of much less than 12 hours.

It is expected that this therapy could reduce the side effects of the traditional limb perfusion therapy as the amount of cytotoxic pharmaceutical compositions needed could, potentially, be drastically reduced. More importantly, this therapeutic strategy would also be a good starting point to investigate the in vivo reaction of high-glycolytic (hypoxic) cancer cells to blood glucose control, as the “troublesome” brain is then fully bypassed.

A suggested procedure could involve performing pre- and post-dialysis PET scans to find the percentage metabolic-active cancer cells after treatments at different blood glucose levels and different treatment periods.

In yet another disclosed embodiment of the proposed treatment and installation, the brain could be separately fed during glucose (and glutamine and other metabolic energy sources) restriction treatment. Such an installation therefore protects the glucose supply of the brain to ensure metabolism of the brain independently of the glycaemic state in the rest of the patient's body.

First, the brain or cerebral glucose circuit is monitored and glucose administered accordingly to ensure that the brain's glucose environment is controlled at a specific set point (optionally within the normoglycaemic range). Cerebral glucose levels should therefore be maintained to at least 0.6 μmol/g, equivalent to a blood glucose level of 6 mmol/l (patient-specific), (Gruetter et al. 1998, Poitry-Yamate et al. 2009). This blood glucose level corresponds to plasma glutamine levels of 0.9-1.35 mmol/l (Kaadige et al. 2009, Patel 2004), which should also be maintained.

Blood glucose supply to the brain could be accomplished using a transcatheter arterial approach. This technique is frequently and successfully performed in, for example, transcatheter arterial chemoembolization (or TACE), (Alba et al. 2007). As with any interventional procedure, there is however risk (approximately 4%) of haemorrhage and/or damage to blood vessels (Levy et al. 2009, Siegel et al. 1998).

Second, as other treatment regimes might be applied to the body, the remainder of the patient's body (defined as the systemic circulatory circuit, excluding the cerebral circulatory network) might be in a state of severe hypoglycaemia. Blood flow to the brain would be independent from that of the rest of the body in this proposed two-zone set-up. This implies that the body-zone dialysis of glucose could approach 0 mmol/l, as was the case with the already discussed treatment of a localised limb.

In still another disclosed embodiment, which may be used with all previously disclosed embodiments that involve the treatment of cancerous proliferative disorders, the dialysis membrane may be coated with functionalized TRAIL (Tumour Necrosis Factor (TNF) Related Apoptosing-Inducing Ligand) and E-selectin surface, as described in King et al. (2009) and Rana et al. (2008). This would facilitate circulating cancer cells being given apoptopic signals, locally.

### RELATED APPLICATION DATA

Continuation of PCT/IB2008/055112, abandoned. Continuation of South African preliminary patents, application no. 2010/00801 and 2009/08784.

### REFERENCES CITED

US PATENT DOCUMENTS

| | | | |
|---|---|---|---|
| 5,851,985 | December 1998 | Tepic, S. et al. | Treatment of tumours by arginine |
| | | | deprivation. |
| 2010/0099726 | April 2010 | Cantley, L. et al. | Inhibitors of pyruvate kinase and |
| | | | methods for treating disease. |
| 2008/0063637 | May 2008 | Tsichlis, P. N. et al. | Regulation of oncogenes by Akt- |
| | | | specific isoforms. |
| 2010/0197612 | August 2010 | Ko, Y. H. | Composition and methods for the |
| | | | treatment of cancer. |
| 2005/0214268 | September 2005 | Cavanagh, W. A. et al. | Methods for treating tumors and |
| | | | cancerous tissue. |
| 2003/0069200 | April 2003 | Platt, M. F. et al. | Use of glucosylceramide synthesis |
| | | | inhibitors in brain cancer therapy. |
| 2010/0014637 | January 2010 | Duma, C. | Leading-edge cancer treatment. |
| 4,303,636 | December 1981 | Gordon, R. T. | Cancer treatment. |
| 2003/0228568 | December 2003 | Bucala, R. J. et al. | Inducible phosphofructokinase and |
| | | | the Warburg effect. |
| 2006/0128777 | June 2006 | Bendall, H. H. | Cancer treatments. |
| 2008/0319054 | December 2008 | Kun, E. et al. | Treatment of cancer. |
| 5,069,662 | December 1991 | Bodden, W. L. | Cancer treatment. |
| 2003/0125283 | July 2003 | Gatenby, R. A. | Therapy of proliferative disorders by |
| | | | direct irradiation of cell nuvlei with |
| | | | tritiated nuclear targeting agents. |
| 2002/0193313 | December 2002 | Bagheri, K. | Method for increasing the efficacy of |
| | | | chemotherapy in the treatment of tumors. |
| 7,582,619 | September 2009 | Von Borstel, R. W. | Compositions and methods for treatment |
| | | | of mitochondrial diseases. |
| 2009/0226427 | September 2009 | Belligand, J.-L. et al. | Novel pharmaceutical compositions for |
| | | | modulating angiogenesis. |
| 2010/0075947 | October 2009 | Aftab, D. T. et al. | Methods of using PI3K and MEK |
| | | | modulators. |
| 4,370,983 | February 1983 | Lichtenstein, E. S. | Computer-control medical care system. |
| 7,338,461 | March 2008 | Linde, P. G. | Modified organ support devices |
| 4,191,182 | March 1980 | Popovich, R. P. et al. | Method and apparatus for continuous |
| | | | plasmapheresis. |
| 3,946,731 | March 1976 | Lichtenstein, E. S. | Apparatus for extracorporeal treatment |
| | | | of blood. |
| 2010/0266589 | October 2010 | Hedrick, C. et al. | Adjuvant cancer therapy. |
| 2010/0143324 | June 2010 | Mononen, I. et al. | Method for treatment of cancers or |
| | | | inflammatory diseases. |
| 5,646,185 | July 1997 | Giaccia, A. J. et al. | Tumor treatment method. |
| 2009/0169591 | July 2009 | Kaul, G. et al. | Medical articles for the treatment of |
| | | | tumors. |
| 2005/0208023 | September 2005 | Krissansen, G. W. et al. | Tumor treating composition and methods. |
| 2003/0113764 | June 2003 | Bodary, S. C. et al. | Compositions and methods for the |
| | | | treatment of tumors. |
| 2005/0136502 | June 2005 | Riser, B. L. et al. | Treatment of disorders associated with |
| | | | elevated blood glucose or blood pressure. |
| 2003/0017995 | January 2003 | Khalifah, R. J. et al. | Dialysis solutions and methods. |
| 2006/0035844 | February 2006 | Ito, F. et al. | Preventive or remedy for diseases caused |
| | | | by hyperglycemia. |
| 2003/0045582 | March 2003 | Wuerth, J.-P. et al. | Method for treating hyperglycemia. |
| 2007/0021357 | January 2007 | Tobia, A. et al. | Treatment of inflammatory conditions. |
| 4,861,485 | August 1989 | Fecondini, L. | Haemodiafiltration device |
| 7,758,533 | July 2010 | Matson, J. R. | Hemofiltration systems, methods and devices |
| | | | for treatment of chronic and acute diseases. |
| 2010/0114002 | May 2010 | O'Mahoney, J. et al. | Method and apparatus for an extracorporeal |
| | | | control of blood glucose. |
| 2007/0135750 | June 2007 | Kraemer, M. | Blood treatment device. |
| 2005/0274672 | December 2005 | Tu, H. et al. | Extracorporeal pathogen reduction system. |
| 2001/0039392 | November 2001 | Strahilevitz, M. | Extracorporeal affinity adsorption methods |
| | | | for the treatment of atherosclerosis, cancer, |
| | | | degenerative and autoimmune diseases. |
| 2009/0139930 | June 2009 | Sternby, J. et al. | Extracorporeal blood cleaning. |
| 5,277,820 | January 1994 | Ash, S. R. | Device and method for extracorporeal |
| | | | blood treatment. |
| 7,604,739 | October 2009 | Huang, T.-N. | Dialysis device. |
| 2010/0111908 | May 2010 | Lin, F. et al. | Induction of renal cells for treatment |
| | | | of kidney disease. |
| 4,702,829 | October 1987 | Polaschegg, H.-D. et al. | Hemodiafiltration apparatus. |
| 6,610,027 | August 2003 | El Hatu, M. K. M. | Hemodialysis. |
| 5,518,623 | May 1996 | Keshaviah, P. et al. | Hemodialysis monitoring system for |
| | | | haemodialysis machines. |
| 3,441,136 | April 1969 | Serfass, E. J. et al. | Controlled blood dialysis system. |
| 5,536,412 | July 1996 | Ash, S. R. | Hemofiltration and plasmafiltration |
| | | | devices and methods. |

### OTHER PUBLICATIONS

Adamian, M., et al. Cutting balloon angioplasty for the treatment of in-stent restenosis: a matched comparison with rotational atherectomy, additional stent implantation and balloon angioplasty, Journal of the American College of Cardiology 2001; 38(3): 672-679.
Agardh, C.-D., et al. Hypoglycemic brain injury, Acta Neurapathol (Berl) 1980; 50:31-41.
Alba, E. et al. Transcatheter arterial chemoembolization in patients with hepatocellular carcinoma on the waiting list for orthotopic liver transplantation. AJR 2007; 190:1341-1348.
Aoki, J., et al. FDG PET of primary benign and malignant bone tumors: standardized uptake value in 52 lesions, Radiology 2001; 219(3):774-777.
Arauz, A., et al. Carotid plaque inflammation detected by ¹⁸F-fluorodeoxyglucose-positron emission tomography: pilot study, Clin. Neurol. Neurosurg. 2007; 109(5):409-412.
Auer, R. N. Hypoglycemic brain damage. Foren. Sci. Int 2004; 146:105-110. Available at http://www.ncbi.nlm.nih.gov/pubmed/20950942
Avril, N., et al. Metabolic characterization of breast tumors with positron emission tomography using F-18 fluorodeoxyglucose, J. Clin. Oncol. 1996; 14:1848-1857.
Ayre, S. G., Perez Garcia y Bellon, D. & Perez Garcia, D. IPT: a new concept in the management of chronic degenerative disease. Med. Hypoth. 1986; 20:199-210.
Balinsky, D., Platz, C. E. & Lewis, J. W. Enzyme activities in normal, dysplastic, and cancerous human breast tissues. J. Natl. Cancer Inst. 1984; 72:217-224.
Balkwell, F., & Mantovani, A. Inflammation and cancer: back to Virchow? The Lancet 2001; 357:539-545.
Bonvalot, S. et al. Hyperthermic isolated limb perfusion in locally advanced soft tissue sarcoma and progressive desmoids-type fibromatosis with TNF 1 mg and melphalan (T1-M HILP) is safe and efficient. Ann. Surg. Oncol. 2009; 16:3350-3357.
Bosch, J. P. & Stein, J. H. Hemodialysis, high-efficiency treatments. Churchill Livingstone Inc., New York, 1993.
Brahimi-Horn, M. C., Chiche, J., & Pouysségur, J. Hypoxia and cancer. J Mol Med 2007; 85:1301-1307.
Briscoe, D. A., Fiskum, G., Holleran, A. L. & Kelleher, J. K. Acetoacetate metabolism in AS-30D hepatoma cells. Mol. Cell Biochem. 1994; 136:131-137.
Byström, P. et al. Early prediction of response to first-line chemotherapy by sequential [18F]-2-fluoro-deoxy-D-glucose positron emission tomography in patients with advanced colorectal cancer. Ann. Oncol. 2009; 20:1057-1061.
Cahill, G. F. et al. Hormone-energy source interrelationships during fasting. J. Clin. Invest. 1966; 45: 1751-1769.
Cahill, G. F. Energy source Metabolism in Starvation. Annu. Rev. Nutr. 2006; 26:1-22.
Casazza, J. P., Felver, M. E. & Veech, R. L. The metabolism of acetone in rat. J. Biol. Chem. 1984; 259:231-236.
Cascini, G. L. et al. 18F-FDG PET is an early predictor of pathologic tumor response to preoperative radiochemotherapy in locally advanced rectal cancer. J. Nucl. Med. 2007; 47: 1241-1248.
Champe, P. C. and R. A. Harvey (eds.). Lippincott's Illustrated Reviews: Biochemistry. Lippincott Williams and Wilkins, Baltimore, Md., 2008.
Chen, C. Y.-C. Inhibiting the vascular smooth muscle cells proliferation by EPC and DPCC liposomes encapsulated magnolol, J. Chinese Inst. Chemical Engineers 2008; 39(5):407-411.
Chi, V. D. & Semenza, G. L. Oncogenic alterations of metabolism. TIBS 1999; 24:68-72.
Chin, B. B., Green, E. D., Turkington, T. G., Hawk, T. C. & Coleman, R. E. Increasing uptake time in FDG-PET: Standardized Uptake Values in normal tissues at 1 versus 3 h. Mol. Imaging Biol. 2008; 11:118-122.
Choi, I.-Y., Lee, S.-P., Kim, S.-G. & Gruetter, R. In vivo measurements of brain glucose transport using the reversible Michaelis-Menten model and simultaneous measurements of cerebral blood flow changes during hypoglycemia. J Cereb. Blood Flow Metab. 2001; 21: 653-663.
Cryer, P. E. Hypoglycemia, functional brain failure, and brain death. J. Clin. Invest. 2007; 117:868-870.
Dang, C. V. & Semenza, G. L. Oncogenic alterations of metabolism. TIBS 1999; 24:68-72.
Dastidar, S. G. & Sharma, S. K. Activities of glycolytic enzymes in rapidly proliferating and differentiated C6 glioma cells. Exp Cell Biol 1989; 57:159-164.
Daugirdas, J. T., Blake, P. G. & Todd, S. I. Handbook of Dialysis, 3rd ed. Lippincott, Williams and Wilkins, Philadelphia, 2001.
Davies, J. R., et al. FDG-PET can distinguish inflamed from non-inflamed plaque in an animal model of atherosclerosis. Int. J. Cardiovasc. Imaging 2010; 26:41-48.
DeBerardinis, R. J. & Cheng, T. Q's next” the diverse functions of glutamine in metabolism, cell biology and cancer. Oncogene 2010; 29:313-324.
DeBerardinis, R. J., Lum, J. J., Hatzivassiliou, G. & Thompson, C. B. The biology of cancer: metabolic reprogramming that energy sources growth and proliferation. Cell Metab. 2000; 7: 11-20.
Dehdashti, F., et al. Positron Tomographic assessment of estrogen receptors in breast cancer: comparison with FDG-PET and in vitro receptor assays, J. Nucl. Med. 1995; 36(10):1766-1774.
Delbeke, D. et al. Evaluation of benign and malignant hepatic lesions with positron emission tomography. Arch. Surg. 2009; 133:510-516.
Eggermont, A. M. M., De Wilt, J. H. W. & Ten Hagen, T. L. M. Current uses of isolated limb perfusion in the clinic and a model system for new strategies. Lancet Oncol. 2003; 4:429-437.
Evans, J. M., Donnely, L. A., Emslie-Smith, A. M., Alessi, D. R. & Morris, A. D. Metformin and reduced risk of cancer in diabetes patients. BMJ 2005; 330:1304-1305.
Fanciulli, M. et al., 1993. Glucose metabolism in adriamycin-sensitive and -resistant LoVo human colon carcinoma cells. Oncol Res 1993; 5:357-362.
Feron, O. Pyruvate into lactate and back: from the Warburg effect to symbiotic energy energy source exchange in cancer cells. Rad. Oncol. 2009; 92:329-333.
Fidler, I. J. The pathogenesis of cancer metastasis: the ‘seed’ and ‘soil’ hypothesis revisited. Nature Rev. Cancer 2003; 3:1-6.
Garber, K. Energy deregulation: licensing tumors to grow. Science 2006; 312:1158-9.
Gatenby, R. A. & Gillies, R. J. Why do cancers have high aerobic glycolysis? Nature Rev. Cancer 2004; 4:891-899.
Gatenby, R. A. A change of strategy in the war on cancer. Nature 2009; 459:508-509.
Gerstein, H. C. Does insulin therapy promote, reduce, or have a neutral effect on cancers? JAMA 2010; 303:446-447.
Geschwind, J. F., Georgiades, C. S., Ko, Y. H. & Pedersen, P. L. Novel therapy for liver cancer: direct intra-arterial injection of a potent inhibitor of ATP production. Cancer Res. 2002; 62:3909-3913.
Gray, L. H. et al. The concentration of oxygen dissolved in tissues at the time of irradiation as a factor in radiotherapy. Br. J. Radiolog. 1953; 26:638-648.
Gruetter, R., Ugurbil, K. & Seaquist, E. R. Steady-state cerebral glucose concentrations and transport in the human brain. J. Neurochem. 1998; 70:397-408.
Grünhagen, D. et al. Isolated limb perfusion with tumor necrosis factor and melphalan prevents amputation in patients with multiple sarcomas in arm or leg. Ann. Surg. Oncol. 2005; 12:1-7.
Guhlmann, A., et al. Fluorine-18-FDG PET and Technetium-99m antigranulocyte antibody scintigraphy in chronic osteomyelitis, J. Nucl. Med. 1998; 39 (12): 2145-2152.
Guppy, M., Geriner, E. & Brand, K. The role of the Crabtree effect and an endogenous energy source in the energy metabolism of resting and proliferating thymocytes. Eur. J. Biochem. 1993; 212:95-99.
Hanahan, D. & Weinberg, R. A. The hallmarks of cancer. Cell 2000; 100:57-70.
Hamdy, R. C. Paget's disease of the bone, Clinics in Geriatrics Medicine 1994; 10(4):719-735.
Helmlinger, G. Interstitial pH and PO₂ gradients in solid tumors in vivo: High-resolution measurements reveal a lack of correlation. Nature Med 1997; 3:177-182.
Howell, A., Chapman, M., & Harvie, M. Energy restriction for breast cancer prevention. In Cancer Prevention II (Senn, H.-J., Kapp, U., and Otto, F., eds.), Springer-Verlag, Berlin, 2009.
Huang, S.-C. Anatomy of SUV, Nucl. Med. Biol. 2000; 27:643-646.
Ilvesmaki, V. et al. Insulin-like growth factors (IGFs) and their receptors in adrenal tumours: high IGF-II expression in functional adrenocortical carcinomas. J. Clin. Endocrin. Metab. 1993; 77:852-858.
Jadvar, H., Ye, W., Groshen, S. & Conti, P, S. [F-18]-fluorodeoxyglucose PET-CT of the normal prostate gland. Ann Nucl. Med. 2008; 22:787-793.
Jain, K. J. A new target for tumor therapy. New Eng. J. Med. 2009; 360:2669-2671.
Jelluma, N. et al. Glucose withdrawal induces oxidative stress followed by apoptosis in glioblastoma but not in normal human astrocytes. Mol Cancer Res 2006; 4:319-330.
Jones, R. G. & Thompson, C. B. Tumor suppressors and cell metabolism: a recipe for cancer growth. Genes and Dev. 2009; 23:537-548.
Kaadige, M. R., Looper, R. E., Kamalanaadhan, S. & Ayer, D. E. Glutamine-dependent anapleurosis dictates glucose uptake and cell growth by regulating MondoA transcriptional activity. PNAS 2009; 106:14878-14883.
Kallinowski, F., Schlenger, K. H., Kloes, M., Stohrer, M. & Vaupel, P. Tumor blood flow: the principal modulator of oxidative and glycolytic metabolism, and of the metabolic micromilieu of human tumor xenografts in vivo. Int. J. Cancer 1989; 44:266-272.
Kenny, L. M. et al. Quantification of cellular proliferation in tumor and normal tissues of patients with breast cancer by 18F Fluorothymidine-positron emission tomography imaging: evaluation of analytical methods. Cancer Res. 2005; 65:10104-10112.
Kim, J. W. & Dang C. V. Cancer's molecular sweet tooth and the Warburg effect. Cancer Res. 2006; 66:8927-8930.
King, M. R., Western, L. T., Rana, K., & Liesveld, J. L. Biomolecular surfaces for the capture and reprogramming of circulating tumor cells. J. Bionic Eng. 2009; 6:311-317.
Kitano H. Cancer as a robust system: implications for anticancer therapy. Nature Rev. Cancer 2004; 4:227-235.
Kohn, E. C., & Liotta, L. A. Molecular insights into cancer evasion: strategies for prevention and intervention, Cancer Res. 55: 1856-1862.
Komar, G. et al. Decreased blood flow with increased metabolic activity: a novel sign of pancreatic tumor aggressiveness. Clin. Cancer Res. 2009; 15:5511-5517.
Kosaka, N. et al. 18F-FDG PET of common enhancing malignant brain tumours. AJR 2008; 190:W365-W369.
Kroemer, G. & Pouyssegur, J. Tumor cell metabolism: cancer's Achilles' heel. Cancer Cell 2008; 13:472-482.
Kundu, J. K., and Surh, Y.-J. Inflammation: gearing the journey to cancer, Mutations Research/Reviews in Mutations Research 2008; 659:15-30.
Lee, J. L. et al. Glucose deprivation-induced cytoxicity and alterations in mitogen-activated protein kinase activation are mediated by oxidative stress in multipharmaceutical composition-resistant human breast carcinoma cells. J Biol. Chem. 1997; 273:5294-5299.
Levy, D. S., Salter, M. M., & and Roth, R. E. Postoperative irradiation in the prevention of keloids, American Journal of Roentgenology 1976; 127(3):509-510. Levy, J., Brown, E., Daley, C. & Lawrence, A. Oxford Handbook of Dialysis, 3rd edition, Oxford University Press, 2009.
Libby, P., Schwartz, D., Brogi, E., Tanaka, H., Clinton, S. K. A cascade model for restenosis; A special case of atherosclerosis progression. Circulation 1992; 86(6 Suppl):11147-52.
Lichtenstein, L. Histiocytosis X (Eosinophilic Granuloma of Bone, Letterer-Siwe Disease, and Schüller-Christian Disease), Journal of Bone and Joint Surgery, 1964; 46:76-90.
Lin, X. et al. 2-Deoxy-D-glucose-induced cytotoxicity and radiosensitization in tumor cells is mediated via disruptions in thiol metabolism. Cancer Res. 2003; 63:3413-3417.
Löffler, M. et al. High diagnostic value of ¹⁸F-FDG-PET in pediatric patients with chronic inflammatory bowel disease, Annals of the New York Academy of Sciences 2006; 1072:379-385.
Lowrie, O. H., et al. Diversity of metabolic patterns in human brain tumors: enzymes of energy metabolism and related metabolic energy sources and cofactors. J Neurochem 1983; 41:994-1010.
Mankoff, D. A. & Bellon, J. R. Positron emission tomography imaging of cancer: glucose metabolism and beyond. Semin. Radiat. Oncol. 2001; 11:16-27.
Mankoff, D. A. et al. Blood flow metabolism mismatch: good for the tumor, bad for the patient. Clin. Cancer Res. 2009; 15:5294-5296.
Mantis, J. G., Centeno, N. A., Todorova, M. T., McGowan, R. & Seyfried, T. N. Management of multifactorial idiopathic epilepsy in EL mice with caloric restriction and the ketogenic diet: role of glucose and ketone bodies. Nutr. and Metab. 2004; 1:11 pages.
Martin D S, Spriggs D & Kroutcher J A. A concomitant ATP-depleting strategy markedly enhances anticancer activity. Apoptosis 2001; 6:125-131.
Mathews E H, Liebenberg L, and Pelzer R. High glycolytic cancers and their interplay with the body's glucose demand and supply cycle, Medical Hypotheses, 2010: 9 pages, doi:10.1016/j.mehy.2010.09.006.
Menendez, J. A. & Lupu, R. Fatty acid synthase and the lipogenic phenotype in cancer pathogenesis. Nature Rev. Cancer 2007; 7:763-777.
Molina, M. et al. Glutamine transport by vesicles isolated from tumour-cell mitochondrial membrane. Biochem. J. 1995; 308:629-633.
Moreno-Sánchez, R., Rodríguez-Enríquez, S., Marin-Hernández, A. & Saavedra, E. Energy metabolism in tumor cells. FEBS J. 2007; 274:1393-1418.
Murray, R. K., Granner, D. K. & Rodwell, V. W. (eds.). Harper's Illustrated Biochemistry, 27th ed. (McGraw-Hill) New York, 2006.
Osame, M. et al. Chronic progressive myelopathy associated with elevated antibodies to human T-lymphotropic virus type I and adult T-cell leukemia-like cells, Annals of Neurology, 1987; 21(2):117-122.
Öz, G. et al. Human brain glycogen content and metabolism: implications on its role in brain energy metabolism. Am. J. Physiol. Endocrinol. Metab. 2007; 292:E946-E951.
Öz, G. et al. Human brain glycogen metabolism during and after hypoglycemia. Diabetes 2009; 58:1978-1985.
Pan, J. W., Rothman, D. L., Behar, K. L., Stein, D. T. & Hetherington, H. P. Human brain—hydroxybutyrate and lactate increase in fasting-induced ketosis. Cereb. Blood Flow Metab. 2000; 20:1502-1507.
Patel, A. B. et al. Glutamatergic neurotransmission and neuronal glucose oxidation are coupled during intense neuronal activation. J. Cereb. Blood Flow 2004; 24:972-985.
Poitry-Yamate, C., Lei, H. X. & Gruetter, R. The rate-limiting step for glucose transport into the hypothalamus is across the blood-hypothalamus interface. J. Neurochem. 2009; 109(Suppl):38-45.
Pollak, M. Insulin and insulin-like growth factor signaling in neoplasia. Nature Rev. Cancer 2009; 8:915-928.
Rana, K., Liesveld, J. L., & King, M. R. Delivery of apoptopic signal to rolling cancer cells: a novel biomimetic technique using immobilized TRAIL and E-selectin. Biotech. and Bioeng. 2008; 102:1692-1702.
Riccardi, V. M. et al. Neurofibromatosis: Phenotype, Natural History and Pathogenesis, Plastic & Reconstructive Surgery 1987; 91(3):561.
Sahlmann, C. O., et al. Dual time point 2-[¹⁸F]fluoro-2′-deoxyglucose positron emission tomography in chronic bacterial osteomyelitis, Nuclear Medicine Communications 2004; 25(8):819-823.
Schiepers, C. & Hoh, C. K. Positron emission tomography as a diagnostic tool in oncology. Eur. Radiol. 1998; 8:1481-1494.
Seyfried, T. N. & Mukherjee, P. Targeting energy metabolism in brain cancer: review and hypothesis. Nutr. and Metab. 2005; 2:30.
Seyfried, T. N. & Shelton L. M. Cancer as a metabolic disease. Nutr. and Metab. 2010; 7:7.
Seyfried, T. N. Does the existing standard of care increase glioblastoma energy metabolism?The Lancet 2010:2 pages, doi:10.1016/S1470-2045(10)70166-2.
Seyfried, T. N., et al. metabolic management of brain cancer. Biochimica et Biophysica Acta 2010: 18 pages, doi: 10.1016/j.bbabio.2010.08.009.
Shankar, L. K. et al. Consensus recommendation for the use of 18F-FDG PET as an indicator of therapeutic response in patients in National Cancer Institute trials. J. Nucl. Med. 2006; 47:1059-1066.
Shaw, R. J. Glucose metabolism and cancer. Curr. Opin. Cell Biol. 2006; 18:598-608.
Shelton, L. M., et al. Calorie restriction as an anti-invasive therapy for malignant brain cancer in the VM mouse. ASN NEURO 2(3):171-177, doi:10.1042/AN20100002.
Shen, J., et al. determination of the rate of the glutamate/glutamine cycle in the human brain by in vivo ¹³C NMR, Proc. Natl. Acad. Sci. USA 1999; 96:8235-8240.
Siegel, G. J., Agranoff, B. W., Fisher, S. K., Albers, R. W. & Uhler, M. D. (eds.) Basic neurochemistry: molecular, cellular and medical aspects, 6th ed. Lippincott-Raven Publishers, Philadelphia, 1998.
Simons, A. L., Matson, D. M., Dornfeld, K. & Spitz, D. R. Glucose deprivation-induced metabolic oxidative stress and cancer therapy. J. Cancer Res. Ther. 2009; 1:S2-S6.
Son, J. M., et al. Varying degrees of FDG uptake in multiple benign neurofibromas on PET/CT, The British Journal of Radiology 2007; 80:e222-e226.
Souhami, R. & Tobias, J. Cancer and its management. Blackwell Publishing, Malden, Mass., 2005.
Suh, W. S., et al. Hypoglycemic neuronal death is triggered by glucose reperfusion and activation of neuronal NADPH oxidase, J. Clin. Invest. 2007; 117(4): 910-918.
Tateishi, U. et al. Bone metastasis is patients with metastatic breast cancer: morphologic and metabolic monitoring of response to systemic therapy with integrated PET/CT. Radiol. 2008; 247:189-196.
Teli et al. Determinants of progression to cirrhosis or fibrosis in pure alcoholic fatty liver. Lancet 1995; 346(8981):987-90.
Tennant, D. A., Duran, R. V. & Gottlieb, E. Targeting metabolic transformation for cancer therapy. Nature Rev. Cancer 2010; 10:267-277.
Van Loon, L. J. C., Greenhaff P. L., Constantin-Teodosiu, D., Saris, W. H. M. & Wagenmakers, A. J. M. The effects of increasing exercise intensity on muscle energy source utilisation in humans. J. Physiol. 2001; 536:295-304.
Vander Heiden, M. G., Cantley, L. C. & Thompson, C. B. Understanding the Warburg Effect: The Metabolic Requirements of Cell Proliferation. Science 2009; 324:1029-1033.
Vaupel, P. Tumor microenvironmental physiology and its implications for radiation oncology. Sem. Rad. Oncol. 2004; 14:198-206.
Vaupel, P., Kallinowski, F. & Okunieff, P. Blood flow, oxygen and nutrient supply, and metabolic microenvironment of human tumors: a review. Cancer Res. 1989; 49; 6449-6465.
Vousden, K. H. & Ryan, K. M. p53 and metabolism. Nature Rev. Cancer 2009; 9:691-700.
Vriens, D, et al. benign uterine uptake of FDG: a case report and review of literature, The Journal of Medicine 2010; 68(9):379-380.
Wahren, J., Felig, P., and Hegenfeldt, L. Physical exercise and fuel homeostasis in diabetes mellitus, Diabetologia 1978; 14:213-222.
Wahren, J., Felig, P., Ahlborg, G., and Hegenfeldt, L. Glucose metabolism during leg exercise in man, J. Clin. Invest. 1971; 50:2715-2725.
Walsh, N. P., et al. Glutamine, exercise and immune function: links and possible mechanisms, Sports Medicine 1998; 26(3):177-191.
Wang, Y., Chiu, E., Rosenberg, J. & Gambhir, S. S. Standardized Uptake Value atlas: characterization of physiological 2-deoxy-2-[18F]fluoro-D-glucose uptake in normal tissue. Mol. Imaging Biol. 2007; 9:83-90.
Wang, Y., Chiu, E., Rosenberg, J. & Gambhir, S. S. Standardized Uptake Value atlas: characterization of physiological 2-deoxy-2-[18F]fluoro-D-glucose uptake in normal tissue. Mol. Imaging Biol. 2007; 9:83-90.
Warburg, O. On the origin of cancer cells, Science 1956; 23:309-314.
Weber, W. A. et al. Positron emission tomography in non-small cell lung cancer: prediction of response to chemotherapy by quantitative assessment of glucose use. J. Clin. Oncol. 2003; 21:2651-2657.
Wheatley, D. N., Campbell, E., Lai, P. B. S. & Cheng, P. N. M. A rational approach to the systemic treatment of cancer involving medium-term depletion of arginine. Gene Ther. Mol. Biol. 2005; 9:33-40.
Wise, D. R. et al. Myc regulates a transcriptional program that stimulates mitochondrial glutaminolysis and leads to glutamine addiction. PNAS 2008; 105:18782-18787.
Wong, C. O. et al. Correlating metabolic and anatomic responses of primary lung cancers to radiotherapy by combined F-18 FDG PET-CT imaging. Rad. Oncol. 2007; 2.
Yuneva, M., Zamboni, N., Oefner, P., Sachidanandam, R., Lazebnik, Y. Deficiency in glutamine but not glucose induces MYC-dependent apoptosis in human cells. J Cell Biol 2007; 178:93-105.
Zasadny, K. R. & Wahl, R. L. Standardized Uptake Values of normal tissues at PET with 2-[fluorine-18]-fluoro-2-deoxy-D-glucose: variations with body weight and a method for correction. Radiol. 1993; 189:847-850.
Zhao, S., et al. Dynamic MET PET analysis has an additional value for differentiating tumors from granulomas: an experimental study using small animal PET, J. Nucl. Med. 2009; 50(Suppl. 2):1650.
Zhuang, H., et al., Dual time point ¹⁸F-FDG PET imaging for differentiating malignant from inflammatory processes, J. Nucl. Med. 2001; 42(9):1412-1417.
Zirngibl, H., Husemann, B. & Hermanek, P. Intraoperative spillage of tumor cells in surgery for rectal cancer. Dis. Colon Rectum 2005; 33:610-614.
Zu, X. L. & Guppy, M. Cancer metabolism: facts, fantasy, and fiction. Biochem. and Biophys. Res. Comm. 2004; 313:459-465.
Zuccoli, G., et al. Metabolic management of glioblastoma multiforme using standard therapy together with a restricted ketogenic diet: case report. Nutrition & Metabolism 2010; 7:33.

1. A haemodialysis machine retrofit and control installation, comprising:
an intake-flow blood glucose sensor, connectable to the blood intake-flow of the haemodialysis machine;
an intake-flow blood glutamine sensor, connectable to the blood intake-flow of the haemodialysis machine;
a return-flow blood glucose sensor, connectable to the blood return-flow of the haemodialysis machine;
a return-flow blood glutamine sensor, connectable to the blood return-flow of the haemodialysis machine;
a dialysate glucose controller for controlling the glucose concentrations in the dialysate;
a dialysate glutamine controller for controlling the glutamine concentrations in the dialysate;
a central control unit, connected to the blood glucose sensors, the blood glutamine sensors, the dialysate glucose controller and to the dialysate glutamine controller for regulating the glucose and glutamine levels in the dialysate to obtain a required blood glucose concentration at the return-flow blood glucose sensor and a required blood glutamine concentration at the return-flow blood glutamine sensor; and
an electroencephalograph (EEG) monitor providing the central control unit with information pertaining to spontaneous electro-cerebral activity to initiate raising of glucose and glutamine levels.

2. The haemodialysis machine retrofit installation of claim 1, further comprising a multi-dimensional concentration sensor for sensing any one or more of a concentration of ketone bodies, glutamine, insulin, hydrogen ions, and urea, measurement of dialysate and blood, blood conductivity, the multi-dimensional concentration sensor connectable into the blood intake flow of the haemodialysis machine, with sensor outputs connected to the central control unit.

3. The haemodialysis machine retrofit installation of claim 1, further comprising a pharmaceutical compound infusion module connectable into the blood return-flow of the haemodialysis machine, with its control inputs connected to an output of the central control unit.

4. The haemodialysis machine retrofit installation of claim 1, further comprising a dialysate glucose sensor connectable into the dialysate circuit, with sensor outputs connected to the central control unit.

5. The haemodialysis machine retrofit installation of claim 1, further comprising a regime database connected to the central control unit, the regime database containing treatment regimes for controlling any one or more of the dialysate glucose controller and the pharmaceutical compound infusion module.

6. The haemodialysis machine retrofit installation of claim 5, wherein the regime database defines any one of a pre-defined metabolic energy source concentration and a predefined dose of a pharmaceutical composition for a particular condition.

7. The haemodialysis machine retrofit installation of claim 1, further comprising a patient monitoring unit, the patient monitoring unit operable to monitor any one or more of the following: electroencephalograph (EEG), electrocardiogram (ECG), blood glucose (body), blood glutamine (body), blood ketone (body), cerebral glucose, cerebral glutamine, cerebral ketone, blood pressure, heart rate, and blood flow rates.

8. A cerebral glycaemic control module, comprising:
an extracorporeal circulation circuit, connectable at one end to cerebral arteries and at another end to cerebral veins of a human or animal body;
a blood glucose sensor and a flow rate sensor, connectable to the cerebral artery side of the circulation circuit;
a blood glucose sensor and a flow rate sensor, connectable to the cerebral vein side of the circulation circuit;
a blood glutamine sensor and a flow rate sensor, connectable to the cerebral artery side of the circulation circuit;
a blood glutamine sensor and a flow rate sensor, connectable to the cerebral vein side of the circulation circuit;
a blood ketone body sensor and a flow rate sensor, connectable to the cerebral artery side of the circulation circuit;
a blood ketone sensor and a flow rate sensor, connectable to the cerebral vein side of the circulation circuit;
a pharmaceutical compound infusion module disposed into the extracorporeal circulation circuit;
a multi-metabolic energy source infusion module disposed into the extracorporeal circulation circuit; and
a central control unit, connected to the glucose sensors, glutamine sensors, ketone body sensors, the flow rate sensors and the infusion modules, for controlling the multi-metabolic energy source infusion module.

9. The cerebral glycaemic control module of claim 8, wherein the central control unit is operable to dispense a pre-defined amount of a metabolic energy source into the extracorporeal circulation circuit.

10. A method for the extracorporeal treatment of blood to absolute minimum levels of metabolic energy sources to maintain homeostasis, the method comprising:
receiving blood from an animal or human into a haemodialysis machine retrofitted with a haemodialysis machine retrofit and control installation as claimed in claim 1;
employing on the haemodialysis machine a new pre-determined computer-controlled treatment regime for the systemic removal of metabolic energy sources to a desired level;
controlling the level of metabolic energy sources in the blood over a pre-determined range by means of central control unit and retrofitted dialysis machine by monitoring a patient's spontaneous electro-cerebral activity by electroencephalography (EEG) and receiving feedback to the controller to ensure spontaneous electro-cerebral activity of the patient's brain throughout the treatment; and
returning the blood from the retrofitted haemodialysis machine to the animal or human.

11. The method of claim 10, wherein the pre-determined treatment regime and the central control unit define any one of a pre-defined metabolic energy source concentration and a predefined dose of a pharmaceutical composition for a particular condition.

12. The method of claim 11, furthering comprising lowering concentrations of predefined metabolic energy sources in the blood through manipulation of conventional dialysis according to the pre-determined treatment regime and control to the best possible precision via the central control receiving EEG feedback of the cerebral activity of the brain, so as to protect the brain.

13. The method of claim 11, further comprising raising concentrations of predefined metabolic energy sources in the blood through dialysis according to the pre-determined treatment regime being controlled by the central control unit, and receiving EEG feedback of the cerebral activity of the brain.

14. The method of claim 11, further comprising infusing pharmaceutical compositions into the blood according to the pre-determined treatment regime.

15. The method of claim 11, further comprising administering pro-apoptotic signals via the use of a dialysis membrane which is treated with a pharmaceutical composition comprised of TRAIL (Tumour Necrosis Factor (TNF) Related Apoptosing-Inducing Ligand) and E-selectin.

16. The method of claim 10, further comprising sensing concentrations of compounds in the blood via the central control unit to direct the execution of the pre-determined treatment regime, the compounds in the blood being selected from any one or more of glucose, glutamine, and ketone bodies, and by monitoring the patient's spontaneous electro-cerebral activity by electroencephalography (EEG) and providing feedback to the controller to ensure spontaneous electro-cerebral activity of the patient's brain.

17. A method of treating a proliferative disorder in a human or animal, comprising:
reducing via a haemodialysis machine retrofitted with a haemodialysis machine retrofit and control installation as claimed in claim 1 any one or both the blood glucose concentration and the glutamine concentration in the human or animal body for a pre-defined period of time to a minimum threshold level as indicated by the onset of abolition of spontaneous electro-cerebral activity as monitored by electro-encephalography (EEG) signals in the control program;
suppressing the blood glucose counter-regulation demand mechanism in the human or animal body; and
suppressing the rate of hepatic glucose production mechanism in the human or animal.

18. The method of claim 17, further comprising controlling the blood glucose concentration in the human or animal body to a level of 2 mmol/l or lower.

19. The method of claim 17, further comprising controlling the blood glutamine concentration in the human or animal body to a level of 0.3 mmol/l or lower.

20. The method of claim 17, wherein the blood glucose counter-regulation mechanism in the human or animal body is suppressed by administering benzodiazepines as initiated by the central control unit.

21. The method of claim 17, wherein the hepatic glucose production mechanism in the human or animal body is suppressed by administering biguanide-class pharmaceutical compositions as initiated by the central control unit.

22. The method of claim 17, further comprising the prior step of subjecting a human or animal body to dietary restriction of 400 to 500 kcal per day by administering a high-ketogenic diet, thus supplying the human or animal body with ketone body compounds to maintain the ketone body concentrations to between 0.8 mmol/l and 1.6 mmol/l, controlled via the central control unit.

23. The method of claim 22, further comprising controlling the blood glucose concentration in the human or animal body to lower than 2 mmol/l while monitoring the patient's EEG activity and providing EEG feedback to the central control unit to control the administration of benzodiazepines, biguanides, and parenteral blood glucose infusion, to ensure spontaneous electro-cerebral activity of the patient's brain.

24. The method of claim 17, further comprising the local treatment of rolling cancer cells by administering pro-apoptotic signals via the use of a dialysis membrane which is treated with pharmaceutical compositions.

25. A method of treating a proliferative disorder in a human or animal, comprising:
isolating the blood circulation system in any one of a limb and an organ of a human or animal body; and
reducing extracorporeally by means of a haemodialysis machine retrofitted with a haemodialysis machine retrofit and control installation as claimed in claim 1 any one or both of the blood glucose concentration and the blood glutamine concentration in any one of the isolated limb and the organ to a blood glucose level of lower than 0.1 mmol/l and to a blood glutamine level of lower than 0.3 mmol/l for a pre-defined period of time.

26. A method of treating a proliferative disorder in a human or animal, comprising:
isolating the cerebral circulation system of a human or animal body from the rest of the blood circulation system;
controlling extracorporeally the glucose concentration in the cerebral circulation system via a new control system to a normal level of between 0.2 μmol/g to 0.4 μmol/g for a pre-defined period of time still ensuring spontaneous electro-cerebral activity as monitored by electroencephalography (EEG); and
controlling the glutamine concentration in the cerebral circulation system to a normal level of between 0.1 μmol/g to 0.3 μmol/g for a pre-defined period of time to maintain spontaneous electro-cerebral activity as monitored by electroencephalography (EEG).

27. The method of claim 26, further comprising reducing extracorporeally the blood glucose concentration in the human or animal body by a haemodialysis machine retrofitted with a haemodialysis machine retrofit and control installation as claimed in claim 1 to between 0.8 mmol/l and 0.1 mmol/l for a pre-defined period of time and suppressing the rate of hepatic glucose production in the human or animal body.

28. The method of claim 27, wherein any one of the rate of glucose demand from the rest of the blood circulation system of the body and of hepatic glucose production in the human or animal body are suppressed by administering via control unit inputs benzodiazepines and biguanide-class pharmaceutical compositions.

29. The method of claim 26, further comprising administering pro-apoptotic signals via the use of a dialysis membrane which is treated with a pharmaceutical composition comprised of TRAIL (Tumour Necrosis Factor (TNF) Related Apoptosing-Inducing Ligand) and E-selectin.
